(19) United States
(12) Patent Application Publication
(10) Pub. No.: US 2017/0238854 A1
(43) Pub. Date: Aug. 24, 2017

(54) WEARABLE SWEAT SENSOR FOR HEALTH EVENT DETECTION

(71) Applicant: Thomas L. Henshaw, Monument, CO (US)

(72) Inventor: Thomas L. Henshaw, Monument, CO (US)

(21) Appl. No.: 15/439,646

(22) Filed: Feb. 22, 2017

Related U.S. Application Data
(60) Provisional application No. 62/298,220, filed on Feb. 22, 2016.

(51) Int. Cl. A61B 5/145; A61B 10/00; A61B 5/00; B01L 3/00; A61B 5/1455

(52) U.S. Cl. CPC A61B 5/14521; B01L 3/502707; B01L 3/502715; B01L 3/50273; A61B 5/1455; A61B 5/6833; A61B 5/7282; A61B 10/0051; A61B 10/0064; B01L 2200/12; B01L 2300/161; B01L 2400/0406; B01L 2300/0877; B01L 2300/165; B01L 2300/087; B01L 2300/0887; B01L 2200/14; A61B 2090/306

(57) ABSTRACT

A wearable medical device for collecting a biofluid and related methods are disclosed. The device has a patch adapted to removably couple to a user's skin. The patch has at least one flow channel, at least one sample chamber, and at least one biomarker detection chamber. The flow channel and/or the sample chamber conducts the biofluid towards the biomarker detection chamber. The sample chamber is adapted to create a high humidity environment adjacent the user's skin. The flow channel comprises a hydrophobic material and a hydrophilic material.

### CROSS-REFERENCE TO RELATED APPLICATIONS

This application claims priority to U.S. Provisional Application No. 62/298,220 filed on Feb. 22, 2016 and entitled “Wearable Sweat Sensor for Health Event Detection, the entire disclosure of which is hereby incorporated by reference for all proper purposes.

### FIELD OF THE INVENTION

The present invention relates generally to a biofluid collection system.

### BACKGROUND OF THE INVENTION

A collection system is needed to reliably capture a small sample volume for health event detection.

### SUMMARY OF THE INVENTION

An exemplary wearable medical device for collecting a biofluid has a patch adapted to removably couple to a user's skin. The exemplary patch has at least one flow channel, at least one sample chamber, and at least one biomarker detection chamber. The sample chamber is adapted to create a high humidity environment adjacent the user's skin. The at least one flow channel has a hydrophobic material and a hydrophilic material. At least one of the sample chamber or the at least one flow channel is configured to conduct the biofluid towards the biomarker detection chamber.

An exemplary method of collecting a biofluid includes removably coupling a wearable medical device for collecting biofluid to a user's skin for a period of time. The exemplary method also includes creating a high humidity environment adjacent the user's skin, and conducting a biofluid through at least one flow channel having a hydrophobic material and a hydrophilic material towards at least one biomarker detection chamber by way of capillary action.

An exemplary method of making a wearable medical device for collecting a biofluid includes providing a patch adapted to removably couple to a user's skin, and forming at least one flow channel in fluid communication with at least one sample chamber and at least one biomarker detection chamber in the patch. The at least one flow channel has a hydrophobic material and a hydrophilic material. At least one of the sample chamber or the at least one flow channel is shaped and positioned to conduct a biofluid towards the biomarker detection chamber. The sample chamber includes a hydrophobic material, and is shaped and positioned to create a high humidity environment adjacent the user's skin.

### BRIEF DESCRIPTION OF DRAWINGS

Various objects and advantages and a more complete understanding of the present invention are apparent and more readily appreciated by reference to the following Detailed Description and to the appended claims when taken in conjunction with the accompanying Drawings wherein:

FIG. 1. is a cross-sectional view of a biofluid sample collector having hydrophobic and hydrophilic layers;

FIG. 2. is a bottom view of the device in FIG. 1;

FIG. 3. illustrates an embodiment of hydrophilic bands on a water impermeable membrane;

FIG. 4. is a bottom view of the hydrophilic bands in FIG. 3;

FIG. 5. is a schematic representation of a biomarker detection chamber integrated into a biofluid sample collection chamber;

FIG. 6. is a schematic of a biopolymer waveguide;

FIG. 7. is a schematic of a streptavidin-biotin aptamer binding in a biopolymer waveguide;

FIG. 8. is a schematic of a light source and photodetector layout with respect to biomarker detection and sweat sample collection chambers;

FIG. 9. is a schematic layout of a removable adhesive patch system;

FIG. 10. Is a schematic of a cross-section of the removable sweat collection module;

FIG. 11. is a schematic of a free-standing sample analysis chamber for biomarker analysis using a removable biofluid collection module and a smartphone for the excitation source, detection, and data post processing;

FIG. 12. is a schematic of a free-standing sample analysis chamber for biomarker analysis using LED or laser LED as the excitation source, a removable biofluid collection module, and a smartphone for detection and data post processing;

FIGS. 13A, B, and C. are feasibility demonstrations of a agarose-gelatin biopolymer waveguide with a top side fluorescence scattering site;

FIGS. 14A and B. illustrate schematics and test results of a agarose-gelatin waveguide loss measurement on an agarose-gelatin waveguide;

FIG. 15. illustrates a fluorescence response of 5′-Dabcyl-Aptamer-Biotin-3′ with FITC aptamer with respect to concentration on a agarose-gelatin waveguide;

FIG. 16. illustrates fluorescence quenching of 5′-Dabcyl-Aptamer-Biotin-3′ with FITC by Interleukin-6 on an agarose-gelatin waveguide; and

FIG. 17. illustrates a quenching titration of a fluorescent aptamer 5′-Dabcyl-Aptamer-Biotin-3′ with FITC by Interleukin-6 on an agarose-gelatin waveguide and tabulated results.

### DETAILED DESCRIPTION

Cardiovascular diseases, including heart failure and heart attack, are the leading cause of death in the United States and countries world-wide. Nearly 71 million Americans are diagnosed with some type of cardiovascular disease (CVD). Over 735,000 people in the United States have heart attacks each year with about 110,000 heart attacks resulting in death (Mozzafarian D., Benjamin E. J., Go A. S., et al., 2015. Circulation 131, e29-e322). Annually, 525,000 people in the U.S. suffer a first-time heart attack, and of those, about 210,000 have recurring heart attacks. It is generally accepted that early detection of many diseases provides the best prospect for successful treatment, lowest cost, and long term survival. Unfortunately, cardiovascular disease has a relatively few number of early warning signals, and thus detection of a developing disease or looming health event is difficult to diagnose.

The diagnostic approaches for cardiovascular disease detection and monitoring have encountered high technical expertise and cost, or encumbered by time consuming technology and visits to a clinic resulting in diagnosis and treatment delays. Conversely, a real time diagnostic can be envisioned that will diagnose and predict a health event that would significantly reduce cost and mortality. In this manner, a facile, continuous, real-time, diagnostic is advantageous to a patient since it allows them to actively follow their health or disease status, their response to therapy, and valuable data feedback to doctors and clinicians. Further, studies have indicated that patients who are knowledgeable about and actively take a role in their healthcare are more likely to positively affect the treatment outcome.

Networked wearable, nonintrusive chemical and biological sensor systems is an emerging technology, and are expected to impact numerous application areas, ranging from health to biodefense to environmental and sports medicine monitoring. These sensors basically operate using a physical, chemical or biological recognition element to detect biological and chemical species, and a transduction mechanism to convert the physical, chemical and biological response to a signal for diagnostic and health monitoring. These sensors, which draw from a wide range of microfluidic, analyte detection, data analysis and transmission technologies, are inherently fast and sensitive, and can operate continuously with real time data reporting. As such, biosensors are poised to impact health care services through delivering economic, wearable, nonintrusive platforms for continuous everyday health, mental and activity status monitoring.

To monitor the health status of an individual, a characteristic chemical or biological substance is unambiguously detected and correlated as a “tracer” of normal biological operation, morbidity, or pharmacological responses to a therapeutic intervention. These substances are often called biological markers or biomarkers, and are used in the diagnosis, prognosis and individualization of treatment of diseases and disorders including cardiovascular disease (CVD), cancer, diabetes, asthma, depression, and others. There are several performance features that determine the viability of a biomarker as a diagnostic aid to patients and doctors. These include: 1) an accurate, reproducible measurement, 2) a measurement that is reasonably low cost with a rapid turnaround time, 3) the information is unique and not already given by clinical assessment, and 4) if concentrations are measureable and will aid in medical evaluation (Morrow, D. A. and de Lemos, J. A., 2007. Circulation 115, 949-52).

Some of the most widely identified heart failure biomarkers for diagnostic purposes are BNP, Galectin-3, sST2, and GDF-15 (Jarolim, P., 2014. Clinical Laboratory Medicine 34, 1-14). Recently, Galectin-3 was detected in saliva and correlated with serum levels. Further, the galectin-3 levels could be differentiated between heart failure and normal control patients (Zhang, X. et al., 2016. Journal of Clinical Pathology; 69:1100-1104). Interleukin-1, 6 and 18 (IL-1, IL-6, IL-18), C-reactive protein, and Tumor Necrosis Factor-merit attention too as they have been correlated with the severity of heart failure pathogenesis, and are believed to be an independent predictor of heart failure outcome (Ridker, P., et al., 2000. Circulation 101, 1767-1772, Cesari, M., et al., 2003. Circulation, 108, 2317-2322, Bozkurt B, et al., 2010. Heart Fail Rev 15, 331-341). Indeed, elevated cytokines levels have indicated a predisposal to various medical conditions including depression, osteoporosis, diabetes, and cardiovascular diseases. The detection and correlation of Interleukin-6 particular in eccrine sweat was closely correlated to the levels in plasma (Marques-Deak, et al., 2006, Cizza, G., et al., 2008).

A majority of the detection techniques to measure biomarkers have used traditional sandwich ELISA, Western blots, radioimmunoas say (RIA), and chromatographic-mass spectrometry methods. Although these approaches are accurate, sensitive and specific, they are not amenable to a personal wearable or home use platform, require a considerable level of expertise to operate. A substantial effort has been given to construct miniaturized wearable biosensor devices that sample a host of biofluids. These devices typically combine micro- and nano-fluidics, protein based enzyme and antibodies recognition elements, and a variety of electrochemical, colorimetric, mass, and optical detection approaches for analyte detection of chemical and biological substances. Protein recognition probes such as antibodies are highly advantageous since they are highly specific and sensitive towards a molecular target. However, they are disadvantageous because they suffer from denaturing issues which significantly reduce operational lifetime. Further, enzyme and antibody probes are burdened with high production cost.

Alternatively, aptamer probes offer robust stable operation with high sensitivity and enhanced selectivity at reduced production cost. Aptamers are single-stranded oligonucleotide or peptide molecules that can specifically bind to a specific target molecule due to their characteristic 3-D conformation. The aptamer dissociation constants K_{d} are typically on the order of a nM or less, and they can be modified for facile surface immobilization with biotin-streptavidin binding. They can be cloned to very high specification, and are highly repeatable through the evolutionary SELEX process. Although the initial discovery costs can be relatively high, commercial production costs are estimated to be $25-$30 per gram after discovery (Valigra, L., 2007. Drug Discovery & Development. Sep. 6, 2007). Since aptamers demonstrate reversible denaturation, the aptamer sensing elements can also be recycled or even discarded based on the low production cost. Therefore, an aptamer based biorecognition device offers many advantages toward creating a selective, sensitive, low cost biorecognition platform.

Serum, plasma, breath, and urine are the most common biofluids for biomarker sensing, and numerous diagnostic sensing techniques have been developed to quantitate these molecules. However, considerable attention has been given recently to the development of sweat and saliva sensing as these fluids contain an abundance of chemical and biological markers that are correlated to those found in blood (Jadoon, J., et al., 2015. International Journal of Analytical Chemistry Volume, Article ID 164974). The advantage of sweat and saliva sensing is that it can be sampled noninvasively on a person with little or no sample preparation, and be integrated into an all-in-one wearable sample collection and sensing device for continuous biomarker diagnostic analysis.

The baseline technology in biomarker monitoring is the sample collection system, and from this architecture the sweat biosensor design follows. Sweat rates and the corresponding sample volumes are considerable less that the sensors using serum or plasma samples. In sweat collection the volume is usually a microliter or less, and thus a collection system is needed to reliably capture the small sample volume. To overcome the small sample volume, traditional techniques such as pilocarpine iontophoresis are used to actively stimulate sweat volume production. Unfortunately, these approaches add complexity to the collection device in terms size and material logistics. In some cases, an allergic reaction due to chemical sensitivity can occur on the skin with this technique. In addition, the chemical and biological targets are significantly lower in concentration than the corresponding serum or plasma analytes. Typical sweat biomarkers concentrations are at the ng/mL level or less. Thus, a sample analysis technique would require highly sensitive on-board diagnostics to determine the low sample concentration. Alternatively, a sample collection system that concentrates the sample would ease the sensitivity burden of the diagnostic system, and allow for more conventional detection systems with higher detection limits.

The following documents are incorporated herein by reference in their entireties for all proper purposes: Bozkurt B, et al., 2010. Heart Fail Rev 15, 331-341; Cesari, M., et al., 2003. Circulation, 108, 2317-2322; Chen, R. T., 1989. SPIE Vol. 1151 Optical Information Processing Systems and Architectures, 60-71; Choi, M. M. F. and Tse, L., 1999; Analytica Chimica Acta 378, 127-134; Jadoon, J., et al., 2015. International Journal of Analytical Chemistry Volume, Article ID 164974; Jarolim, P., 2014; Clinical Laboratory Medicine 34, 1-14; Li, J. J., Fang, X., and Tan, W., 2002. Biochem. Biophys. Res. Commun. 292, 31-40; Manocchi, A. K., et al., 2009. Biotechnol. Bioeng; 103, 725-732; Morrow, D. A. and de Lemos, J. A., 2007. Circulation 115, 949-52; Mozzafarian D., Benjamin E. J., Go A. S., et al., 2015. Circulation 131, e29-e322; Ridker, P., et al., 2000. Circulation 101, 1767-1772; Valigra, L., 2007. Drug Discovery & Development. Sep. 6, 2007; Zhang, X. et al., 2016. Journal of Clinical Pathology; 69:1100-1104.

To make a wearable sweat biosensor viable and robust for every-day personal use, a sweat collection system is provided to alleviate the drawbacks surrounding iontophoresis, small sample volume, and low sample concentration. One approach to mitigate these drawbacks is to a new, simplified sweat collection system that passively accumulates low sweat volumes, and concentrates the low concentration sample for easier detection and post collection analysis. The details of this approach are described below.

This disclosure pertains to a wearable biofluid collection and concentration patch system for the capturing of biomarkers in sweat for use in the prognosis and diagnosis of a health event. The basis of the disclosure is to use microfluidic hydrophilic and hydrophobic channels with permeable membranes to capture and condense perspiration, concentrate the perspiration, and flow the perspiration to a common collection site where it can be accumulated. The disclosure can be also used to collect saliva samples for biomarker analysis. For the purpose of this document, the terms “flow” and “conduct” may be used interchangeably.

The device relates to a passive biofluidic collection system that collects and concentrates disease related biomarkers in a biofluid such as sweat and saliva for the detection of a health event. The collection system may use enhanced microfluidic hydrophilic and hydrophobic channels with permeable membranes to capture and condense perspiration, concentrate the perspiration, and flow the perspiration to a common collection point. This removes the need for additional chemicals and electrical components to actively generate sweat samples, and helps minimize the fabrication costs, complexity, and footprint while maintaining the sample collection performance.

The overall structure of the health event biosensor system may have three integrated modules: a biofluid sample collection module, a biomarker analyzer module, and a wireless data transmission module.

The biofluid sample collector module may include an adhesive patch made of permeable biopolymers, polymer films and textile fabrics. It may have two components: a biofluid sample collection chamber, and a biomarker recognition chamber. The sample collection chamber gathers a liquid biofluid which contains a biomarker analyte of interest. In the case of sweat, the sample collection chamber serves to generate the condensable sweat from skin pores. In the case of saliva, an oral sample is transferred to the sample collection chamber. The sample collection chamber passively and continuously moves the bulk biofluid sample through to the biomarker detection chamber, and out through a porous membrane. The sample collection chamber is made of several alternating hydrophilic and hydrophobic layers that guide the biofluid to a common collection site that adjoins to the biomarker detection chamber. The biomarker detection chamber receives the biofluid from the sample collection chamber, and isolates a specific biomarker from other bulk biofluid (sweat or saliva) constituents.

In some embodiments, the sweat or saliva sample can be sampled and collected in a discrete or batch mode, where the sweat or saliva sample is collected at prescribed time intervals and transferred to an offline self-contained sensor device for patient self-assessment and monitoring at home.

The biomarker detection chamber may be in physical contact with the sample collection chamber. The biomarker detection chamber receives the biofluid from the sample collection chamber, and isolates a specific biomarker from other bulk biofluid (sweat or saliva) constituents. The biomarker detection chamber may have a biomarker recognition or binding site made of an immobilized aptamer beacon embedded on a biopolymer waveguide. The separation of the biomarker from other biofluid constituents is accomplished by forming a biomarker-aptamer complex within the waveguide.

The aptamer beacon may be constructed such that it undergoes a conformational change as a biomarker binds to it. The aptamer beacon also generally contains a fluorophore whereupon a fluorescent signal is generated through the optical illumination of the aptamer-biomarker complex. The optical fluorescent emission signal is proportional to the amount of biomarker present, and thus the biomarker concentration can be determined. Conversely, the aptamer may be constructed such that fluorescence may be quenched upon biomarker binding.

The waveguide may be constructed from disposable biopolymers that allow the biofluid to flow across its pores and reach the aptamer. The waveguide may be constructed such that fluorescence is decoupled from the illumination source by transmitting the signal to the analyzer chamber detector. This enables the detector to receive an aptamer beacon signal that is free form convolution by the illumination source.

The biomarker analyzer module may be a separate but close-coupled fixture external to the biofluid sample collection module. It may have two component parts which include the aptamer illuminator and the waveguide emission detector. The aptamer illuminator excites the fluorophore within the aptamer beacon at a specific wavelength to generate fluorescence. The waveguide emission detector collects the waveguided optical emission signal generated through the binding the aptamer beacon and target biomarker. The biomarker analyzer is may be made of miniaturized commercial component parts including a light source such as light emitting diode or light emitting diode laser, an optical detector such as a photodetector, CCD or CMOS camera, a power supply, and an optical driver and power conditioning circuit.

The optical emission signal generated by the aptamer-biomarker binding may be converted to an electrical signal using an optical detector. The signal is analyzed and the concentration of the biomarker is monitored and logged in continuous, (real-time) mode or in a non-continuous (variable processing or batch) mode. The analyzer module is designed to be close coupled to the biomarker recognition chamber for maximum signal collection. The biomarker analyzer module contains the necessary electronics, power supply, and firmare to operate the biosensor.

The wireless data transmission module may interface with the biomarker analyzer for acquiring and processing the biomarker signal. A software application orchestrates the logging of the spectral emission signal, subsequent data registration, and general post processing data analysis. The data summary is transmitted through a secure and prompt communication to the patient, doctor, and clinician for assessment and status of the patient progress.

Methods and system components that form a biofluid sample collection system that allows diagnostic and prognostic monitoring of biomarkers are also disclosed herein. The system may be made of layered hydrophobic and hydrophilic materials for the passive transportation of biomarkers in sweat or saliva to a centrally and commonly located channel that intersects with a biomarker detection chamber.

In some embodiments, the biofluid sample collection structure can condense, concentrate and direct the flow of the sweat liquid to a centrally and commonly located channel and biomarker detection chamber.

In some embodiments, an oral saliva sample can be deposited onto the biofluid collection chamber and the saliva can be transported to a centrally and commonly located channel and biomarker detection chamber.

In some embodiments, a commercial, standardized oral fluid collection device can be used to deliver the saliva to the biofluid collection structure.

In some embodiments, the biofluid sample collection chamber has a plurality of channels that direct the liquid to a centrally located, common flow channel, and then to a biomarker detection chamber. The channels are formed by creating adjacent areas of hydrophilic material and hydrophobic material. The aqueous based sample migration occurs in the hydrophilic area while the hydrophobic wall confines the liquid to a narrow channel that concentrates the sample. A plurality of flow channels is created by alternating bands of hydrophobic and hydrophilic material.

In some embodiments, the sweat and saliva collection chamber and channels are made from highly manufacturable, producible, low-cost disposable materials such as polyethylene films, polyacrylic films, cotton or polyester fiber.

In some embodiments, sweat and saliva collection flow channels can be formed through printing or coating of the hydrophobic material onto the hydrophilic material, or vice versa.

In some embodiments, the device may envelop the sample collection module into a wearable skin patch device for home or self-monitoring of a person's health status.

In some embodiments, sweat rate and volume can be measured within the biofluid sample collection chamber.

In some embodiments, the biorecognition structure is composed of an aptamer for the selective and sensitive detection of the biomarker in the presence of sample fluid constituents.

In some embodiments, the biomarker recognition site is composed of an aptamer immobilized onto a modifiable surface of a biopolymer waveguide. The aptamer can be attached to the biopolymer waveguide using well known streptavidin-biotin binding, or other common chemical and physical modifications.

In some embodiments, a biorecognition structure is composed of multiple, distinct aptamers for the simultaneous detection of a family of key clinical biomarker groupings so as to clearly define the onset and worsening of health event, and the effectiveness of any therapy against the disease.

Some embodiments include a biorecognition structure that combines optical waveguide and fluorescent aptamer assay elements with the layered hydrophobic and hydrophilic sample collection materials.

In some embodiments, the waveguide is composed of non-hazardous biomaterials such as agarose, gelatin, silk, and chitosan, so as to allow for an inexpensive, disposable substrate, yet still create efficient waveguide performance.

In some embodiments, the optical waveguide can be easily fabricated from inexpensive film deposition techniques such as printing, dip coating, spin coating and knife coating.

In some embodiments, the fluorescent based aptamer beacon is used to create an integrated waveguide. Here fluorescent light is coupled into the sensor waveguide using the immobilized fluorescent aptamer as a light source. This removes the need for additional coupling optics and emission filters, and helps minimize the fabrication costs and footprint while maintaining the sensor performance.

In some embodiments, a biosensor structure is connected to an optical illumination source and detection structure to measure the concentration of the biomarker, and transduce the optical biosensor signal to an electronic signal.

Some embodiments include enveloping the sample collection, biofluid analyzer, and wireless data transmission modules into a wearable skin patch device for the transmission of medical diagnostic and monitoring data to an individual or health care professional.

In some embodiments, the targeted biomarker is found in perspiration. Perspiration can happen in the liquid and vapor form. However, only liquid perspiration has the ability to transport the chemical marker to the collection and detection modules. To create liquid perspiration two key factors need to be present; 1) the body requires cooling activating the perspiration response; 2) the area above the sweating pores requires a humidity high enough to reduce the evaporation rate to be below the sweating rate. The high humidity environment causes the onset of liquid sweat from pores even at low levels of sweating.

High humidity environments can be formed in the microclimate next to the skin by preventing perspiration vapor from escaping the area above the skin. This is easily achieved by having any material layer on the skin that does not allow water vapor transport, such as, polyethylene films or polyacrylic films. There are a wide range of films that prevent water vapor diffusion.

Liquid perspiration formation in the skin pores can then be induced and transported by a patch that covers the skin. This patch is made of a material that prevents water vapor diffusion and contains flow channels that direct the liquid perspiration to a collector section.

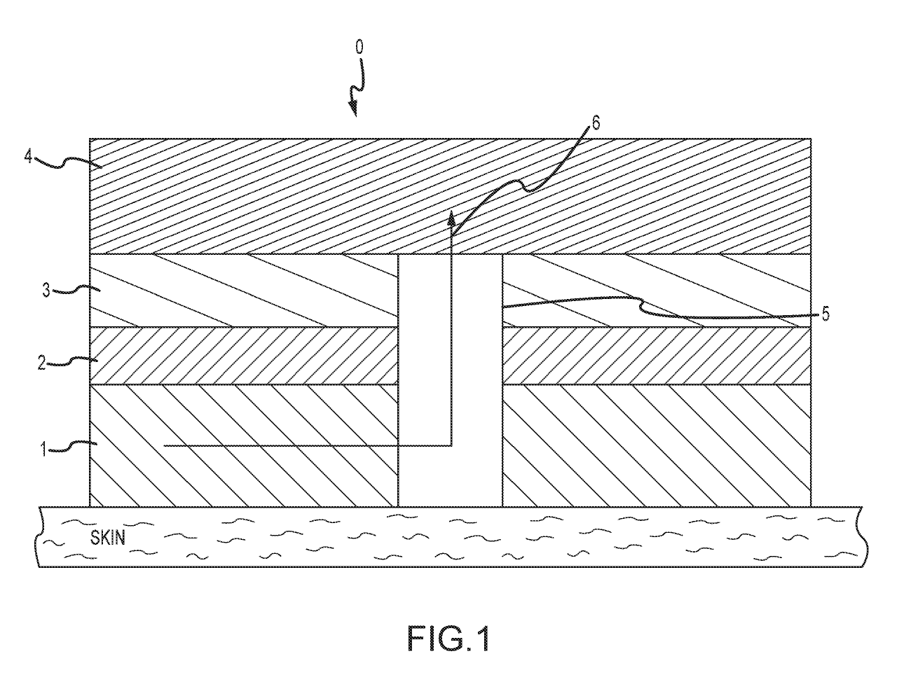

FIG. 1 illustrates a patch configuration of system 0 showing the sweat sample collection chamber elements. The sample collection chamber is a disposable patch that has four stratified hydrophilic and hydrophobic material layers, and a central, common liquid sample flow port. Liquid water transport or wicking occurs in the hydrophilic layers and channels of the sample collection chamber. The hydrophobic material in the sample collection chamber is used to confine water or sweat to defined flow channel area. Layer 1, a hydrophilic material, rests next to the skin. On the basal side (or skin side) of layer 1 is an array of adjacent hydrophilic and hydrophobic microfluidic flow channels that is in contact with the skin. Lying above layer 1 is layer 2, which is a laminated hydrophobic, water impermeable film that prevents water from flowing out through the patch and away from the flow channels. Layer 3 is where the biomarker detection chamber rests. The biomarker chamber is agnostic towards the type of biomarker recognition or binding system, i.e., the chamber is designed to accommodate a broad class of biomarker recognition systems regardless of the manner in which they bind or isolate the biomarker. Layer 4 is a hydrophilic material and is the final layer that lets material flow out of the patch system. The layers are configured in a way so as to create a common central flow port 5 devoid of material. Liquid sample flow 6 collected and channeled in layer 1 flows up towards layer 3 and out layer 4 by capillary action. This 4-layer patch is used to force liquid sweating, direct liquid sweat to a central location and pull liquid through the collection chamber and out on the other side. This allows for the chemical marker to be concentrated at the biomarker detection chamber.

An example of a hydrophilic material such as used in layers 1 and 4 is wicking treated polyester or cotton fabric. An example of a hydrophobic material such as used in layer 2 is a polyacrylic polymer. Other hydrophilic and hydrophobic materials can be used to obtain the same effect.

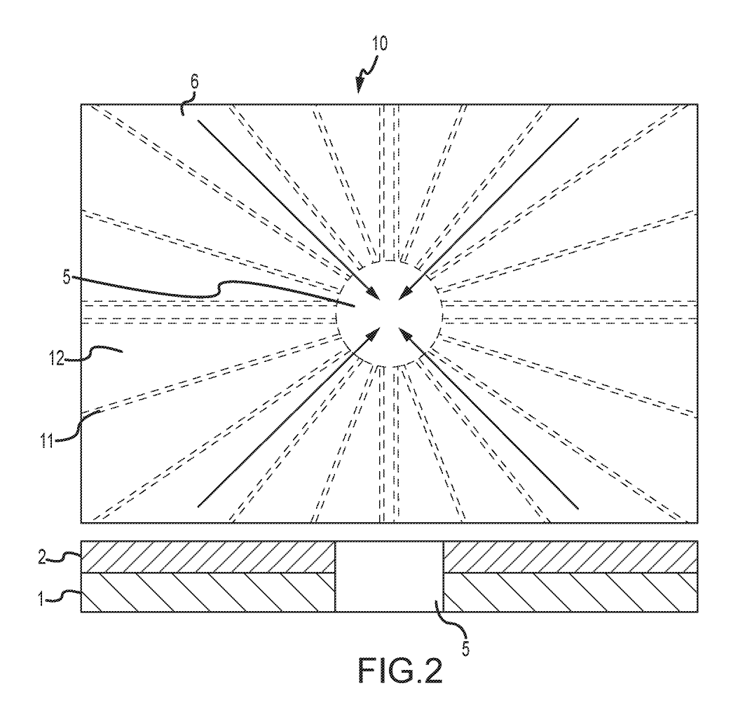

FIG. 2 is a diagram of system 10 showing the liquid flow channel configuration viewed from the basal side of layer 1 in contact with the skin. The liquid flow channels, or wicking channels, are made by creating alternating areas of hydrophilic and hydrophobic material that confine the hydrophilic area to well-defined channel. Pluralities of well-defined liquid flow channel elements 6 are shown, along with the hydrophobic 11 and hydrophilic 12 material bands. The flow channels 6 are created by printing on the bottom side of layer 1 a hydrophobic polyacrylic band 11 on a wicking treated hydrophilic polyester woven fabric 12. In this diagram all of the flow channels 6 intersect and converge at a centrally located sample port 5. The water migration occurs in the hydrophilic area through capillary wicking, and the direction of the liquid flow is given by the arrows. On the opposite side of the polyester woven fabric 12 is a hydrophobic polyethylene film 2 that is laminated to the polyester woven fabric. The central or common sample port 5 is devoid of the polyethylene film 2 so that liquid water flows into and out of the port to the biomarker detection chamber. A side view of the structure 10 shows the lamination of layers 1 and 2, and the central flow port 5. Note that as the flow channel 6 collection grid area contracts and connects to the sample port 5 collection area, a flow area reduction is created. This in turn causes the sample volume to be proportionately reduced, and the analyte concentration to be enriched as it travels through the sample port to the biomarker detection chamber and biomarker recognition site. Further, the contracted area leads to a smaller sample spot size on the biorecognition site and thus improved signal to noise ratio. For example, given a flow channel collection area of 10 cm² and a common sample collection port of 0.01 cm² (a sample port diameter of approximately 1 mm), a 1000× decrease in the area is created. If the collection port diameter were decreased to 100 microns (0.1 mm) the area ratio decrease is 100,000 times. In this manner the effective measurement sensitivity and signal to noise ratio can be significantly increased which enables enhanced detection of low concentration samples. With modern spotting and fabric production techniques it is conceivable that the area ratio could be even higher.

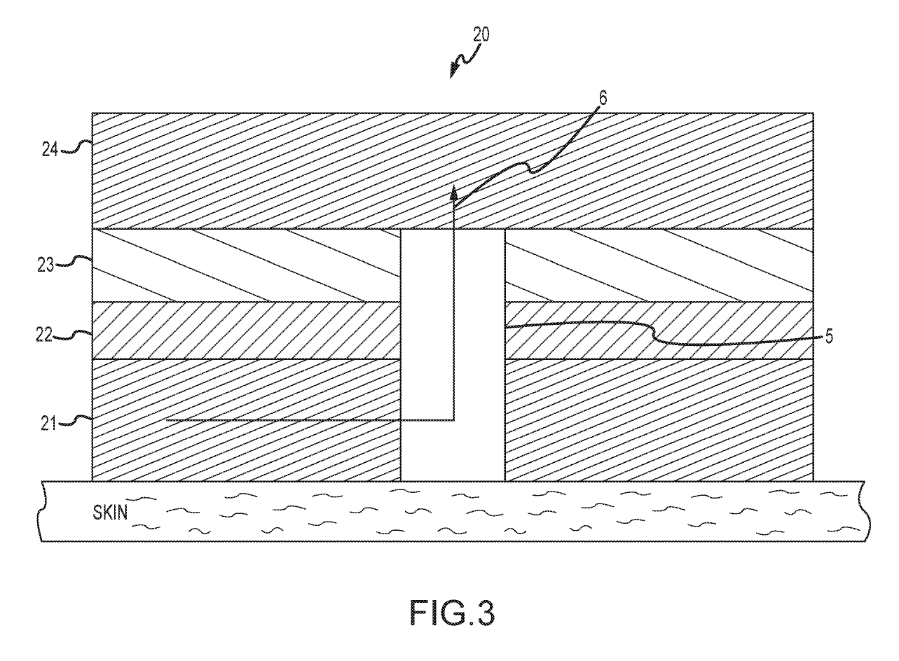

System 20 in FIG. 3 an alternative patch layer system example where hydrophilic bands 21 are printed onto a water vapor impermeable membrane 22. The center portion 5 will again be void of the water vapor impermeable membrane. The biomarker detection chamber 23 is sandwiched between layers 22 and 24. On the opposite side of layer 23 is an outer wicking polyester fabric 24 that covers the membrane 22 and the biomarker detection chamber. The outer fabric 24 is used to aid in evaporating the perspiration and pulling the liquid through the central port.

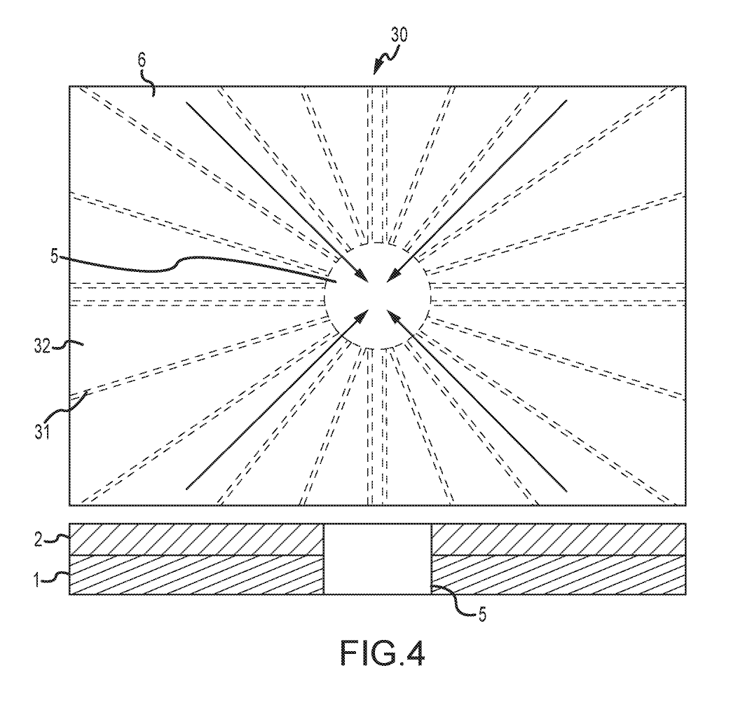

FIG. 4 is a diagram of system 30 showing the alternative flow channel configuration of the underneath side of layer 1. In this configuration hydrophilic, bands 31 are printed onto a water impermeable membrane 32 to create an array of flow channels that guides sweat flow to the central flow port 5. A side view of layers 1 and 2 with the central flow channel is also presented.

Aptamer assays are routinely and readily modified with fluorescent beacons for combined biorecognition and signal transduction. Fluorescent optical detection is a viable approach for a wearable sensor as it provides continuous, sensitive, electromagnetic-free interference mode of detection. Typically, a fluorescent aptamer beacon is created by inserting an aptamer sequence in a molecular beacon-like, hairpin structure that is end-labeled with a fluorophore and a quencher. Photon driven Förster-resonance-energy transfer (FRET) can then be used to construct a “signal-on” mode, or alternatively, a “signal-off” mode. The signal-off mode is advantageous in that they are simpler in designs, and require fewer synthesis steps which can be cost-effective. In a signal-off mode, the fluorescence donor and a quencher are conjugated respectively to both ends of the aptamer. As the biomarker binds to the aptamer, the aptamer undergoes a conformational change which close-couples the donor and quencher, and through a dipole-dipole energy transfer the fluorescence is quenched. For example, a FRET quenching design with fluorescein-DABCYL donor-acceptor aptamer was constructed and demonstrated to analyze thrombin at a detection limit of 373 pM with a K_{d} of 5.20 nM, which is within the realm of required sensitivity for a heart failure biomarker (Li, J. J., Fang, X., and Tan, W., 2002).

The FRET-aptamer beacon is a key element in the sensor concept. The isotropic fluorescence from the immobilized aptamer beacon is coupled into the waveguide without using additional optical components such as fiber optics, gratings or prisms. To initiate the fluorescence, excitation light from a light-source such as a LED or laser diode is absorbed by the aptamer fluorophore in a short optical path length of a few micrometers. Therefore, to achieve high coupling efficiency a fluorophore has to exhibit a high absorption cross-section and good optical stability to couple as much excitation light as possible. Further, to achieve sensitive signal generation the fluorophore should possess a high quantum yield to obtain maximum reemission of light, and a large Stokes shift for prevention of an inner filter effect. Dyes such as Fluorescein or Alexa 488 fulfill these requirements as they exhibit high photon yields, φ>0.90, large absorption cross-sections, σ>1×10⁻¹⁶ cm², and reasonably large Stoke shifts >25 nm, and can be incorporated into the aptamer structure.

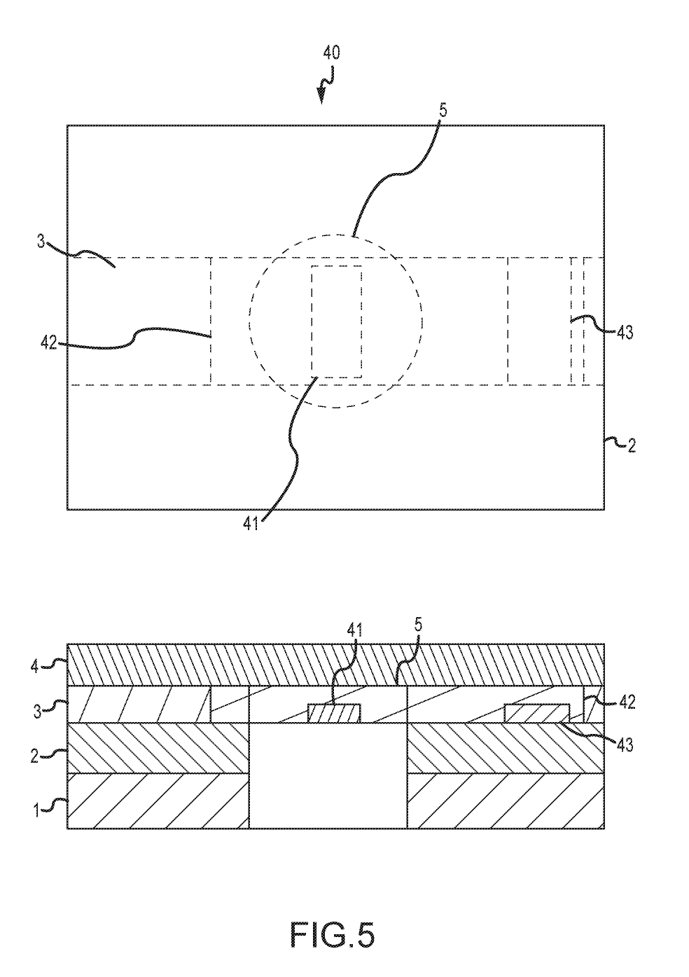

The biomarker detection chamber having the aptamer beacon and waveguide can be integrated into the sweat sample collection chamber. System 40 in FIG. 5 is an illustration of the biomarker detection chamber 3 having the aptamer-based biomarker binding site 41 immobilized onto a biopolymer waveguide sensor 42. The waveguide 42 is composed of simple, inexpensive, non-hazardous biopolymer films such as agarose, gelatin and chitosan. The biomarker detection chamber 3 with biomarker binding site 41 and waveguide 42 comprises a biosensor structure that fits between the sample collection layer 2 and 4, and rests on liquid sample port 5. A waveguide photon scattering or reflection element 43 can be incorporated into the waveguide 42, and is shown downfield of the biomarker binding site 41. The scattering element 43 is used to scatter waveguided light into a photodetector when viewing the guided emission orthogonally from the top or bottom surface of the waveguide. The light scattering can be induced by several means including a notch in the waveguide, grating, or any photoreflective film such as a TiO₂-based polymer film on the waveguide.

Liquid perspiration flows up from layers 1 and 2 and through the sample port 5 into the biomarker detection chamber. The waveguide 42 with immobilized biomarker binding site 41 intersects the liquid flow in sample port 5. At this point the aptamer binds the biomarker analyte in the sweat sample while other sweat constituents are passed through the sample port and into layer 4, and out of sample collection chamber. The top diagram in FIG. 5 shows a view of the biorecognition chamber looking down into the sweat sample chamber to the water impermeable layer 2. The lower diagram shows a cross-section of the overall biorecognition module showing the integration of the biomarker detection chamber in the sweat sample collection chamber.

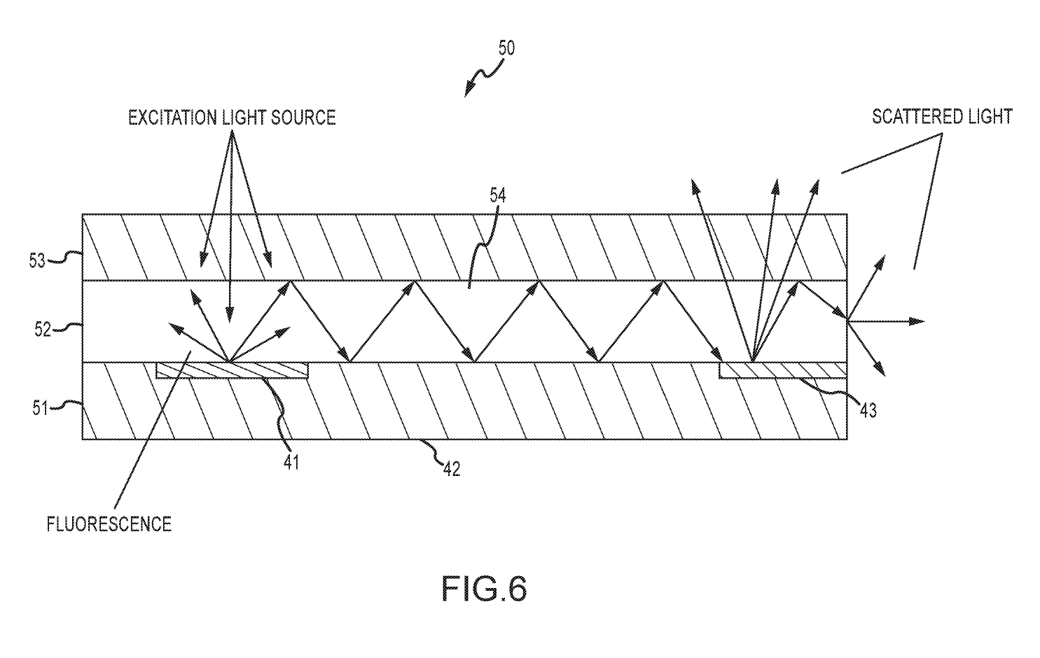

The waveguide can be fabricated from inexpensive, disposable biopolymers such an agarose cladding and gelatin core as illustrated by system 50 in FIG. 6. The waveguide 42 is composed of alternating films of agarose 51, gelatin 52, and agarose 53. The index of refraction for agarose and gelatin films are 1.497 and 1.536, respectively, and are independent of film thickness, concentration, or gelatin Bloom number (Manocchi, A. K., et al., 2009. Biotechnol. Bioeng; 103, 725-732). The waveguide operates as follows. An excitation light source illuminates the FRET-based aptamer beacon 41 in the gelatin layer 52. The resulting fluorescence emission is confined within the gelatin core and is guided by total internal reflection 54 to a remote scattering site on the waveguide 43. At this site the scattered emission can be viewed for detection by a photodetector. Literature reports give an optical loss of <1 dB/cm for this type of waveguide, so the light can be guided a significant distance from the source and detected without the use of an optical filter (Chen, R. T., 1989. SPIE Vol. 1151 Optical Information Processing Systems and Architectures, 60-71, Manocchi, A. K., et al., 2009. Biotechnol. Bioeng; 103, 725-732). The scattered emission can also be viewed at the terminus edge of the waveguide.

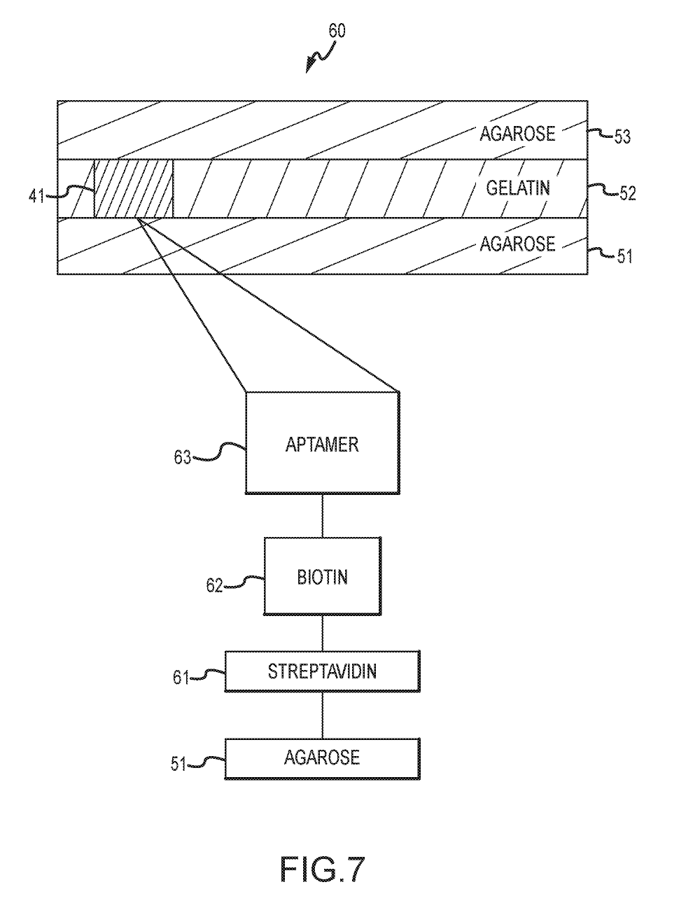

System 60 in FIG. 7 shows how the FRET-based aptamer beacon 41 is deposited in the gelatin 52 layer, and sandwiched between the agarose layers 51 and 53. The top diagram is a side view of the waveguide showing the placement of the aptamer beacon in the gelatin core. A notional breakout of the aptamer binding to the substrate is shown in the lower diagram. The immobilized aptamer waveguide is constructed as follows. First a substrate layer of agarose 51 is made, followed by impregnation of streptavidin 61 on a segment or strip of the agarose substrate 51. Then the biotin 62 portion of the aptamer 63 is immobilized on the streptavidin segment 61. A layer of gelatin 52 is then laid over the immobilized aptamer 63 and agarose film 51. A final layer of agarose 53 is then applied over the gelatin layer 52. In this manner the fluorophore in the aptamer is excited locally within the gelatin layer, and its fluorescence guided within the gelatin layer. Alternatively, the aptamer can be bound onto polystyrene micro-particles using the biotin streptavidin interaction, and then encapsulated into the gelatin layer. Alternatively, thiolated aptamers can be directly attached to amine-modified functional groups of the gelatin.

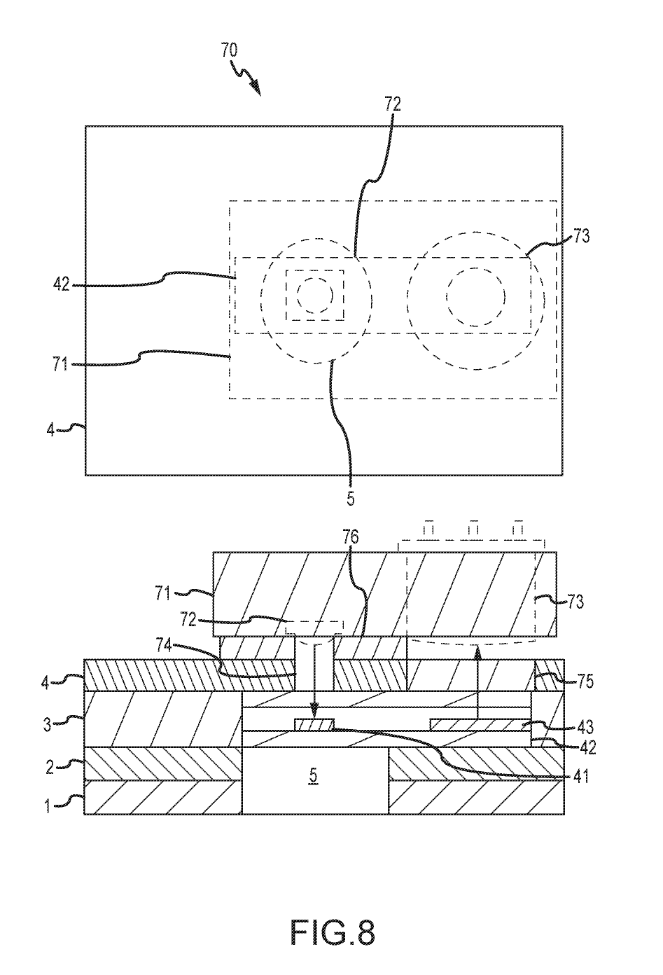

System 70 in FIG. 8 illustrates the positioning of the biomarker analyzer module, which includes the light source and photodetector, with respect to the biomarker detection and sweat sample collection chambers. The upper diagram shows a top view of the light source looking down into the sample sweat collection and biomarker detection chambers. The lower diagram is a side view of the coupling of the light source and photodetector to the sweat sample collection and biomarker detection chambers. The light source 82 and photodetector 83 are contained in a fixture 81 that rests just above layers 1-4 of the sweat collection chamber patch. Light excitation port 84 and photodetection port 85 allow light to enter and leave the waveguide structure 42, respectively. An aperture 86 spatially confines the emission from the light source to the immobilized biomarker binding site 41 on the waveguide. Photodetector 83 is positioned in-line with photodetection port 85 to receive the optical signal from the scattering element 43. The emission signal is collected and transduced into an electrical signal by a photodetector 83. It is then determined if the biomarker concentration in the sweat sample is above or below a determined health risk level.

This data can be sampled and collected continuously or discretely (i.e., in a non-continuous or batch mode), and communicated through a wireless smartphone application or an on-board data collection and data processing center. The acquired fluorescent optical data is used to either generate an on-board signal, or wirelessly using a secure communication protocol to inform the patient, doctor or clinician of the current health status for monitoring the medical condition and treatment.

The entire sweat collection, waveguide, and aptamer biorecognition patch system is packaged into a permanent housing that is wearable on the person's body such as the wrist, back, or other sweat areas. The housing is designed such that it allows for opening and inserting or replacing a patch system as needed. Further, the housing permanently contains all the electrical, excitation light source, photodetector, communication components.

The permanent housing structures, devices, electronics, communications and methods should be apparent to those skilled in the art. These have been previously disclosed without any additional modifications here and should be viewed in the broadest terms.

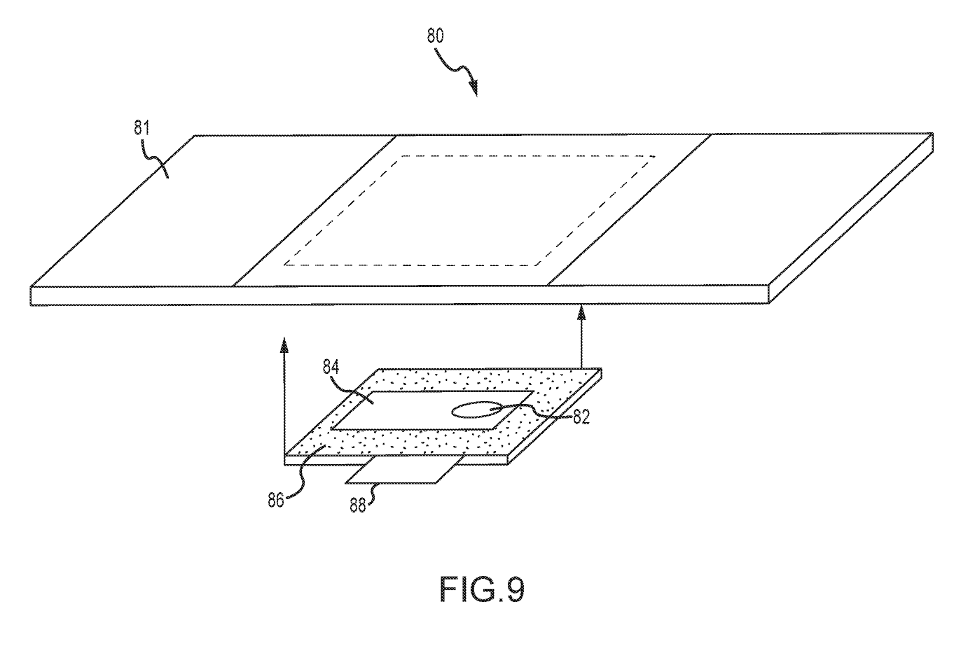

System 80 in FIG. 9 shows the layout of a removable, permeable adhesive patch system that can be applied to bodily sweat areas. It contains a porous adhesive strip 81 that attaches to the skin. On the underneath side a biofluid collection module 86 is attached. This module contains biofluid collection chamber 84 and a biomarker recognition chamber 82. After a prescribed sampling period, the patch 81 is removed from the skin. The patch can be worn in a continuous sampling manner or can be worn in discrete or non-continuous sampling mode. Then the biofluid collection module 86 can be lifted off the porous adhesive strip and placed into separate, free-standing sample analysis chamber for home biomarker analysis. A small lift tab 88 can be used to aid the removal.

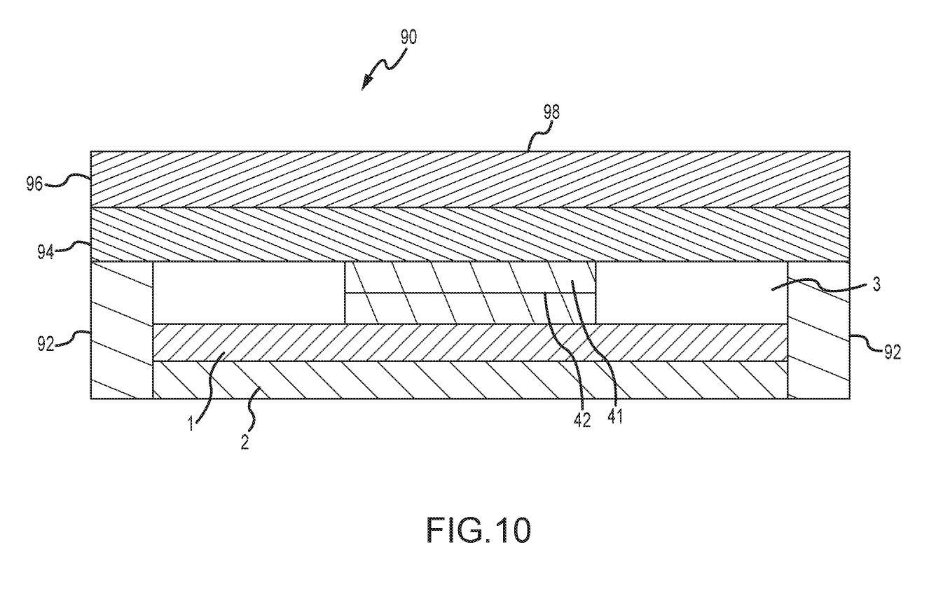

System 90 in FIG. 10 shows a cross-sectional view of the removable adhesive patch system. A layer of polyurethane 94 and a layer of Zod nylon 96 form the outer cover. An adhesive layer 92 is adhered to the polyurethane and Zod nylon to fix the biofluid collection module to the skin. The biomarker detection chamber 3 is formed on the inside of the polyurethane layer and has the aptamer-based biomarker binding site 41 immobilized onto a biopolymer waveguide 42. Below the biorecognition chamber is the biofluid collector chamber which is composed of layer 1, a hydrophilic material, and layer 2, which is a laminated hydrophobic material.

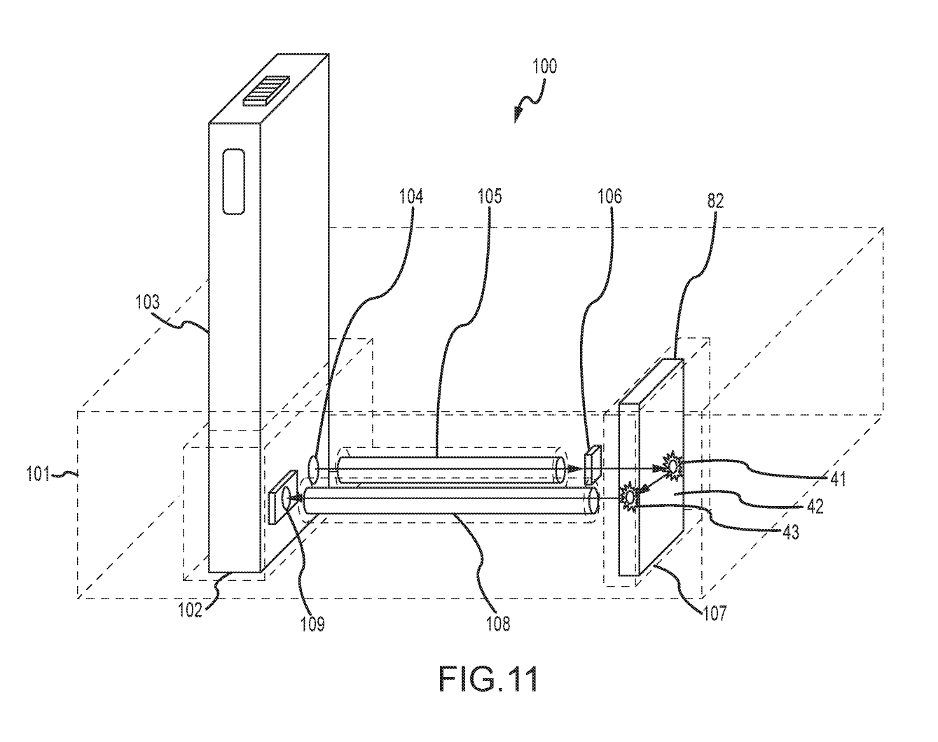

In some cases it is advantageous to have a stand-alone biomarker analysis system to enable an individual to monitor the progress of the biomarker analysis at home or during travel. In FIG. 11 the schematic layout of this approach is given. System 100 contains a detection system that is made of a system support base 101 that securely positions a smartphone 103 into a phone holder slot 102, two optical light guides 105 and 108, and a removable patch 82. The optical light guides has an excitation light pipe 105 and an emission light pipe 108, which are positioned, aligned, and close-coupled to the smartphone flash 104 and camera lens 109 in the system support base. The support base 101 has a patch holder slot 107 for inserting the removable patch 82 containing the biorecognition aptamer 41, biopolymer waveguide 42, and scattering site 43. The light pipe 105 gathers the excitation light from the smartphone flash 103 and directs it to the aptamer biorecognition site 41 on the waveguide 42. An optional filter 106 may be used to narrow the emission spectrum of camera flash. The aptamer biorecognition site 41 generates a fluorescent signal upon excitation, and the waveguide 42 guides the emission to a scattering point 43 downfield of the biorecognition site. The light is then scattered into the emission light pipe 108 which guides the fluorescence to the camera lens 109 and into the camera sensor for analysis.

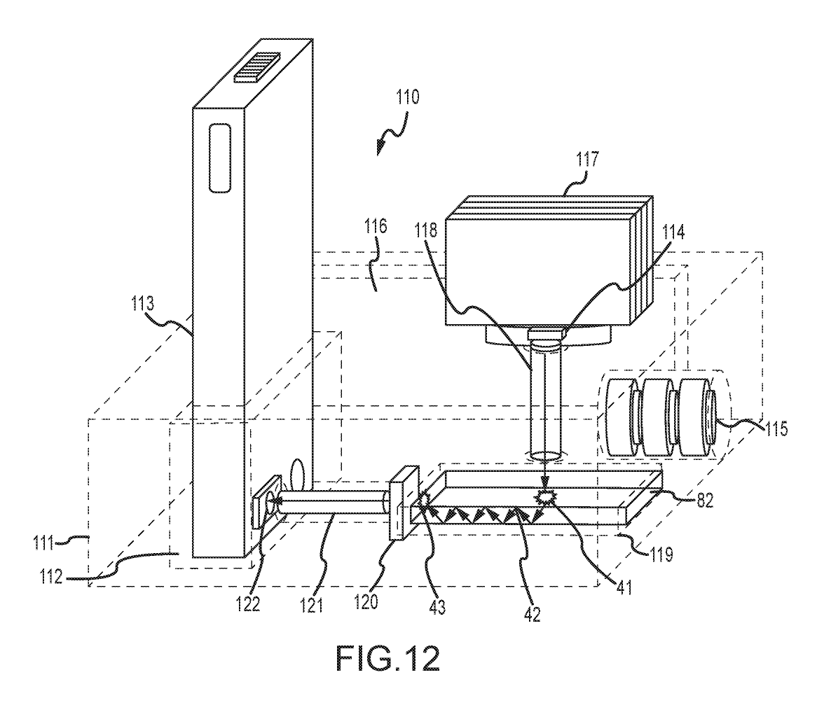

System 110 in FIG. 12 illustrates an alternative layout for a stand-alone biomarker analyzer. The system features are similar to those described in structure 100 except that a separate LED or laser diode excitation source is used. A support base 111 is used to dock the smartphone 113 into a slot holder 112. A LED or laser diode 114 is positioned above the removable patch 82. The LED or laser diode excites the aptamer biorecognition site 41 on the waveguide 42. The LED or laser diode has an electronics control package including a driver circuit 116, power supply 115, and heat sink 117 for controlling the excitation power. For ultra-sensitive detection the use of laser excitation source is preferred. Upon excitation the aptamer 41 generates fluorescence signal which is guide to a scattering site 43 by the biopolymer waveguide 42. To reduce any spurious scattering a long pass emission filter 120 is placed between the waveguide 42 and the emission light pipe 121. This can be an inexpensive long pass filter such as a Kodak Wratten gel filter. The emission light pipe guides the fluorescence into the camera lens 122 for detection and analysis.

Saliva is biofluid medium that has attracted keen interest as numerous biomarkers related to disease signaling have been detected. Saliva composition is mostly water, >99%, with the balance mostly having electrolytes, mucous, antimicrobials, and enzymes. Accordingly, the design structures for the sampling and analysis of sweat are also adaptable to saliva collection in the microfluidic environment. This attributed to the phenomena of flow thinning in microfluid structures, which arises from the alignment of saliva mucines (polysaccharides) with the bulk fluid flow direction.

Saliva flow rate is much higher than sweat flow rates. The unstimulated flow rate is approximately 0.5 mL/min, and stimulated flow rates can reach upward of 3-4 mL/min. In comparison, sweat flow is orders of magnitude less that saliva gland flow. For example, assuming a sweat gland surface density of 200 glands/cm² and a flow rate of 4 μL/min/gland, the sweat flow rate (or flux) is about 0.8 μL/min cm². Thus for a 1 cm² area, the flow rate is 0.8 μL/min. Although saliva sample collection volumes are comparatively robust, the challenge is to capture and deliver consistently a known volume of the saliva to the sample collection chamber. Fortunately, readily available commercial saliva collection technology has advanced significantly to the extent that saliva can be sampled and delivered consistently and reliably.

For the measurement of biomarkers in perspiration the sweat rate and volume are key parameters to characterize the process. However, this measurement can be problematic since the sweat sample area and volume is small. One approach to measuring the sweat rate is to quantify the humidity above the skin at two different heights using two humidity sensors in the sweat patch architecture described here. In this case the difference in the humidity is proportional to the water vapor pressure gradient between the two points from evaporating sweat which allows the sweat rate or flux to be measured. The humidity above the skin can be transduced using a fluorophore immobilized into a gelatin or agarose film as described by Choi and Tse (Choi, M. M. F. and Tse, O. L. 1999. Analytica Chimica Acta 378, 127-134). Thus, the wearable sweat patch architecture developed here can be used for a sweat rate using existing components. For example, the biomarker patch system gelatin waveguide, light source, and detector components can be used for the sweat rate measurement. In lieu of an aptamer beacon, a simple fluorophore like Rhodamine 6G can be used.

The sweat flow rate measurement is performed as follows. Evaporation of water from the patch surface creates a water vapor gradient. The two waveguide films are separated by a known distance and changes in the fluorescent signal, which are are proportional to the concentration gradient, are measured. Assuming the diffusion constant for the water vapor remains constant over the patch height, the sweat flux can be determined. If the measurement is confined to a known area over time, the sample sweat rate and volume can then be determined from the flux.

The following examples illustrate details of some embodiments.

### EXAMPLE 1

### Feasibility of Biopolymer Waveguide with Scattering Site

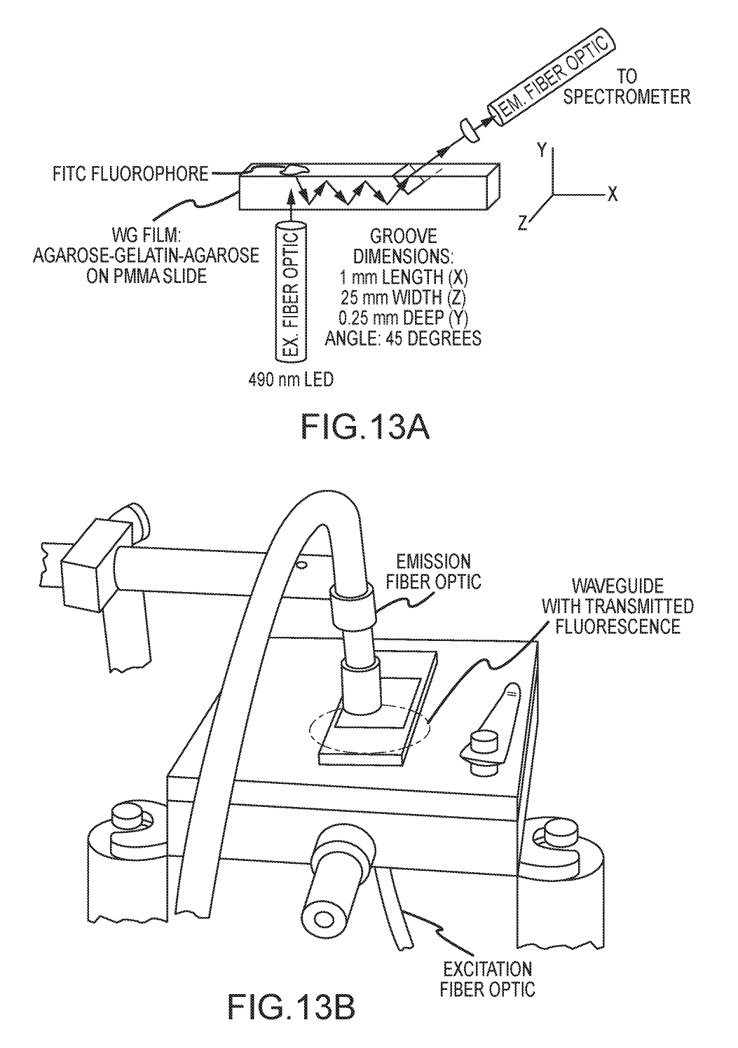

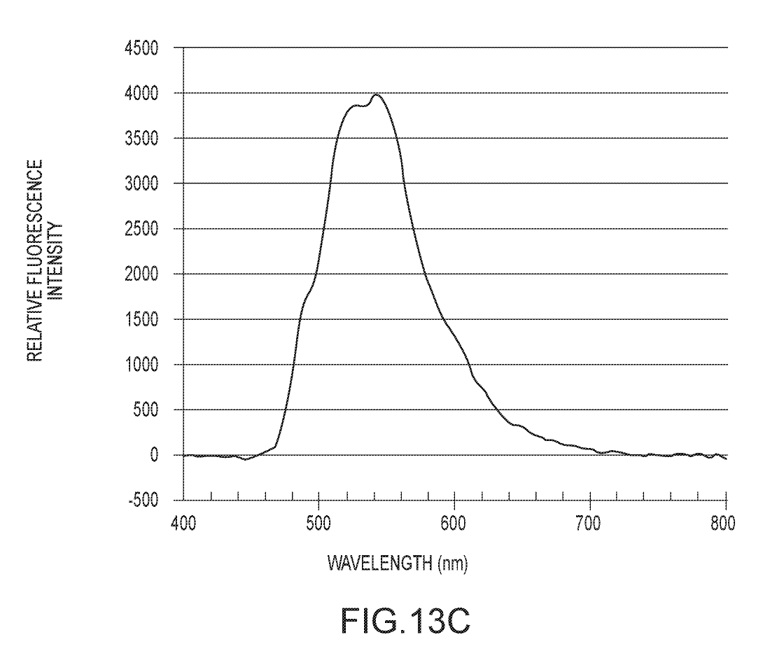

A feasibility demonstration of the biopolymer waveguide with a fluorescence scattering site is shown in FIGS. 13A, B and C. Here an agarose-gelatin waveguide was constructed and deposited onto a Poly(methyl methacrylate) or PMMA substrate. The substrate was 75 mm long×25 mm wide, and 3 mm thick. The waveguide was created using 7.5 wt % agarose as the cladding (300 bloom, η=1.497) and 1 wt % gelatin as the core, (η=1.536) solutions. The agarose and gelatin were prepared in 1× phosphate buffer. The gelatin also contained a small amount mTransglutaminase to cross link it and make it thermally stable and more rigid.

The agarose and gelatin films were crudely hand dip coated in succession onto the slide to create films that were estimated to be less than 50 microns. An angled notch was cut onto its surface at about 45 degrees to scatter light out of the waveguide into a fiber optic so that its emission can be detected by a CCD spectrometer above the waveguide. The notch dimensions are approximately 1 mm length (x), 25 mm width (z), 0.25 mm deep (y). A block diagram of the experimental set-up is shown in FIG. 13A.

A 10 microliter sample of 50 μM fluorescein isothionate (FITC) was spotted onto the film using a hand pipettor. The sample spot was located 2 cm from the grooved notch in the waveguide. The FITC was excited by a 490 nm LED with 1.5 mW optical power at the fiber exit using a multimoded fiber optic (400 μm core, N.A. 0.39). The emission was collected using a 5 mm diameter, f/2 collimator lens with a 10 mm focal length and a multimode fiber optic with a 600 mm core and N.A. of 0.24. The emission fiber optic was then mounted above the waveguide at about 45 degrees and fed to a spectrometer to disperse and record the fluorescence spectra. The waveguide was mounted into an X-Y stage for stability and positioning. The FITC was excited by butt-coupling the excitation fiber optic from underneath the waveguide. The transmitted waveguide emission was collected on the top of the waveguide using the emission collimator mounted fiber optic. A photograph of the setup is shown in FIG. 13B. These are not optimized conditions but are intended to show how the waveguide fluorescence can be scattered out of the waveguide for detection without adding complex alignment optics or filters.

Fluorescent waveguiding was generated, transmitted, and scattered out of the waveguide at a distance of 2 cm notch down field from the sample spot as shown in FIG. 13B. The FITC transmitted emission was collected and dispersed using the spectrometer and CCD camera as shown in FIG. 13C. Note that in a biosensor application the fiber optics used in this demonstration are not be used. Rather, the illuminator and emission detector would be close-coupled to the waveguide. The results of the transmitted fluorescence through the waveguide and its scattering out of the waveguide demonstrate the feasibility of this simple approach. Glass substrates with and without streptavidin coating were also used in place of PMMA substrate and demonstrated the fluorescent waveguiding as well.

### EXAMPLE 2

### Waveguide Loss Measurement

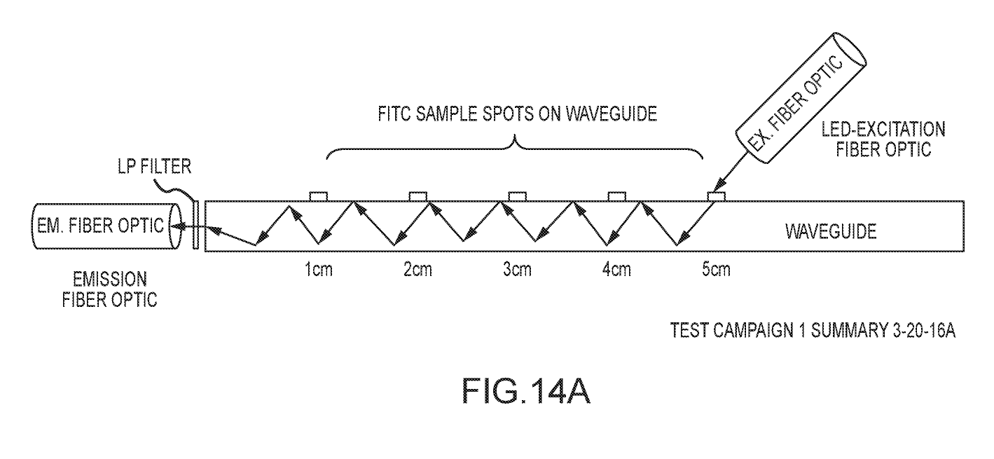

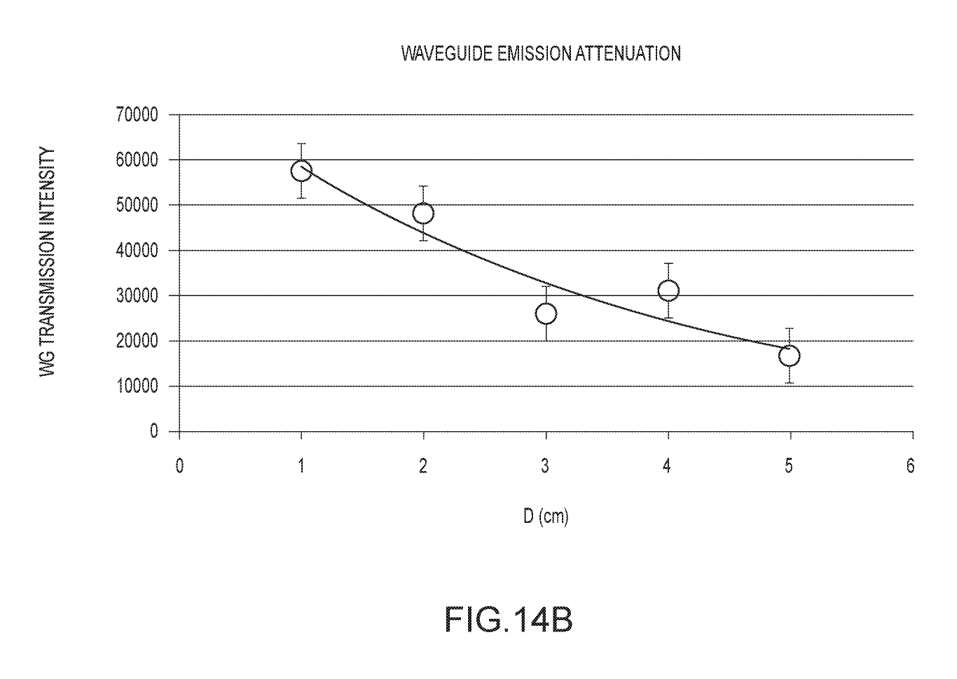

To show that the biopolymer waveguide performance is of sufficient quality to transmit a fluorescent signal down field to a detection point, a waveguide loss measurement was made using an agarose-gelatin waveguide film. The film was made and analyzed in the manner describe in Example 1 except that a 75 mm×25 mm alkyl-amino silanized glass substrate was used and the emission was view at the substrate edge rather than on the top. This approach allowed easier measurement of the emission as a function of distance. The conditions under which the measurement was made are as follows. FITC concentration: 10 μM FITC; FITC sample volume: 5 μL; FITC spot spacing: 5 sample spots separated by 1 cm. The sample spots were delivered using a hand pipettor. FIG. 14A illustrates the measurement setup, and FIG. 14B is the data for the transmitted fluorescence intensity over the 5 cm distance. The loss coefficient, α, can be determined from an exponential fit of the form I(d)=Iₒexp(−αd) to the data, where Iₒ is the initial intensity, α is the loss coefficient, and d is the distance in cm. FIGS. 14A and B shows a fit of the data to this equation, which results in a loss coefficient of −0.29 cm⁻¹ for the waveguide. The error bars in the graph represent the standard deviation of the curve fit to the data. Expressing the loss L in dB/cm, L (dB/cm)=4.3 α, the loss L is −1.25 dB/cm. This comparable to the value of −1 dB/cm obtained by Chen (Chen, R. T., 1989. SPIE Vol. 1151 Optical Information Processing Systems and Architectures, 60-71), and Manocchi (Manocchi, A. K., et al., 2009. Biotechnol. Bioeng; 103, 725-732). These data suggest the separation distance between sample excitation or collection point and the sample detection analysis point should be kept to about 1-2 cm for optimal signal.

### EXAMPLE 3

### Aptamer Fluorescence Response on Waveguide

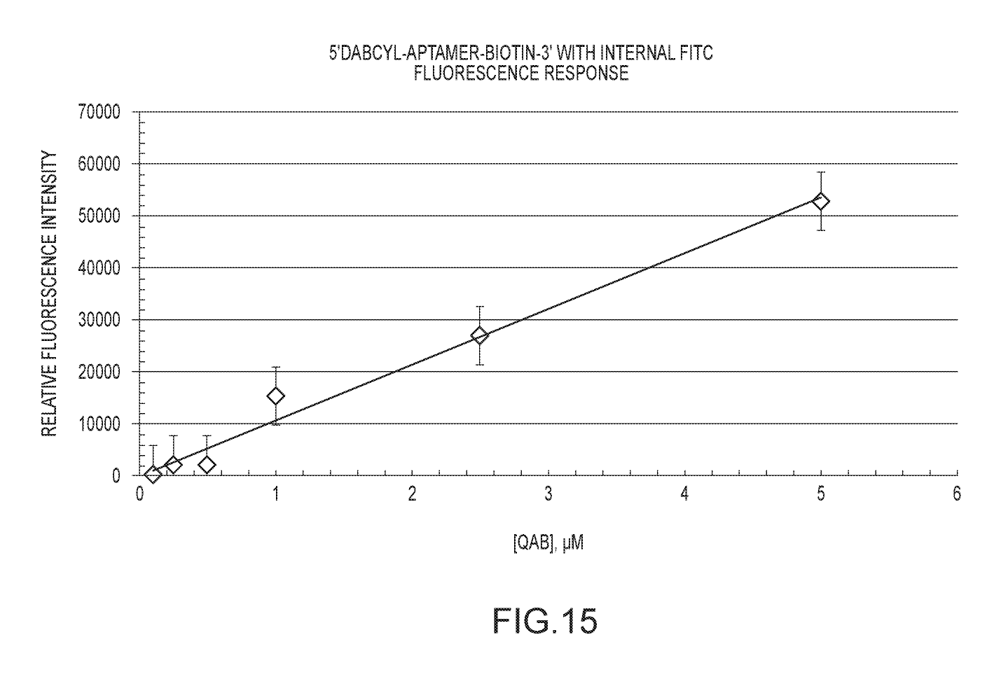

Although the sensitivity of the quenching aptamer beacons (“signal-off” mode) are well known in buffer solutions, the aptamer response in the waveguide environment is less known so as to warrant an investigation. Accordingly, the sensitivity of the fluorescent signal from a quenching aptamer beacon (abbreviated as QAB) with respect to concentration in the waveguide environment was examined. In this demonstration the quenching aptamer beacon used was 5′-Dabcyl-Aptamer-3′-Biotin with internally bound FITC, which was provided by BasePair Biotechnologies (Sequence name ATW0083). The preparation of QAB was followed according to the specification given by BasePair Biotechnologies, Pearland Tex. A variety of QAB concentrations were prepared and diluted in the specified solutions which ranged from 25 nM to 5 μM. A 10 μL aliquot of the QAB solution were then spotted with a pipettor onto waveguide, and emission measurements were taken as a function of concentration. These results are presented in FIG. 15 which shows a linear response over a concentration range of 100 nM to 5 μM. The error bars in the graph represent the standard deviation of the linear regression curve fit to the data. The minimum concentration detected was 25 nM at S/N of 3. However, concentrations less than 25 nM were not reliable as background signals from the waveguide prevented any further analysis. Considering the crude fashion that the waveguide was constructed, this result indicates significant improvement to detection limit can be obtained.

### EXAMPLE 4

### Demonstration Aptamer-IL-6 Fluorescence Quenching on a Waveguide

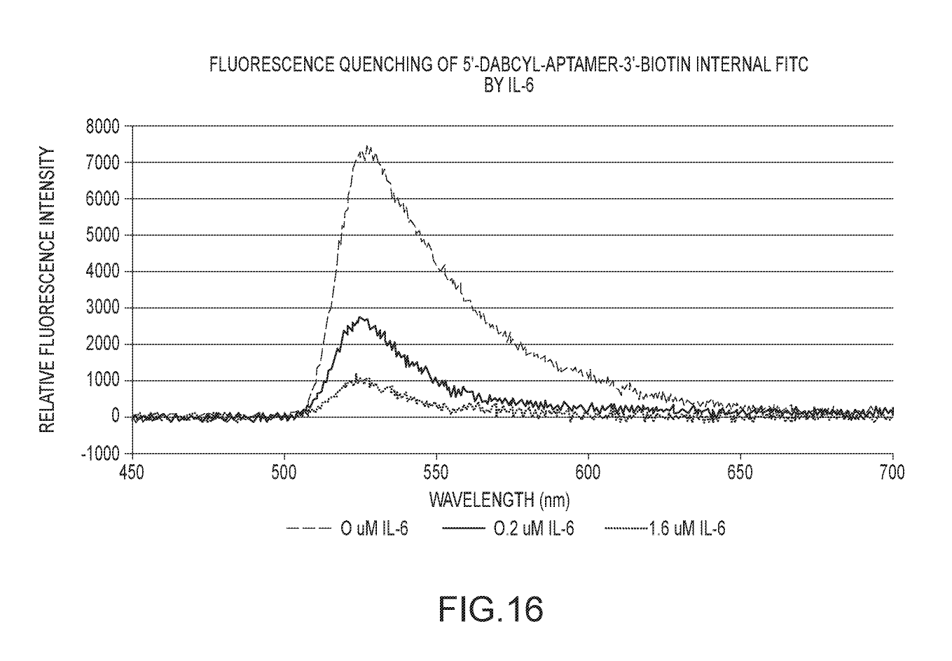

The signal-off mode demonstration of quenching of 5′-Dabcyl-Aptamer-3′-Biotin/FITC by Interleukin-6 (IL-6) on the agarose-gelatin waveguide is shown in FIG. 16. Interleukin-6 is an aptamer target that is a pro-inflammatory cytokine and is implicated as disease biomarker in heart failure. It is present in numerous biofluids including plasma, sweat and saliva. The aptamer is labeled with an efficient amine fluorescent quencher moiety, 4-(dimethylaminoazo) benzene-4-carboxylic acid (Dabcyl) at the 5′ position of the aptamer. Biotin is bound at the 3′ end to dock to streptavidin for immobilization (K_{d}=10⁻¹⁵ M). Within the aptamer, FITC is also labeled which upon illumination produces a visible emission that peaks around 525 nm. The aptamer undergoes conformational structure change (secondary or tertiary folding) when the target molecule IL-6 binds to the aptamer. In doing so, the Dabcyl and FITC are brought together in close proximity to each other so as to undergo Forster Resonance Energy Transfer. In doing so the FTIC fluorescence signal is quenched and thus transduces a signal that is proportional to the concentration of the IL-6 in the sample volume.

IL-6 (Recombinant Mouse IL-6, carrier free) was obtained from BioLegend (Catalog #575706) and prepared according to specifications given by the manufacturer. A 5 μL quenching aptamer beacon sample with a concentration of 1.004 was spotted on the agarose-gelatin waveguide at three different locations. The excitation and emission fiber optics were set up to probe and collect the FITC signal of the aptamer at a distance of 1.5 cm from the sample spot. The aptamer was allowed to dry in a humidity chamber. Then a 5 μL sample of IL-6 at 0, 0.2 and 1.6 μM was then spotted onto the sample with a pipettor. The spot area that formed on the waveguide was about 2 mm in diameter, and each spot was given twenty minutes to stabilize the signal before the fluorescence was captured.

As shown in FIG. 16, the FITC fluorescence decreased upon the addition of higher concentrations of IL-6, indicating the quantitative ability of the aptamer to complex the IL-6 target on the waveguide. The delay in the analysis time to stabilize the signal may possibly be due to combined photo-bleaching effects of the FITC and IL-6 diffusion through the agarose-gelatin waveguide. Still, the time to process the signal is sufficiently fast to enable continuous monitoring. It is also noted that more stable fluorescent labels are available that are much less susceptible to photo-bleaching.

### EXAMPLE 5

### Performance Comparison of the Aptamer-IL-6 Binding on Waveguide to Literature

To determine if the waveguide with the immobilized quenching aptamer beacon performs similarly to that reported in the literature, a quenching titration was conducted using Il-6 as the target biomarker. The data obtained from the titration allows the extraction of the molar binding ratio, r_{b}, between IL-6 with 5′-Dabcyl-Aptamer-3′-Biotin/FITC, and the dissociation constant, K_{d}. These values can then be compared to literature values and an assessment of the biosensor performance can be made.

Each sample had a fixed 5′-Dabcyl-Aptamer-3′-Biotin/FITC concentration, (QAB) with variable IL-6 concentration. The QAB and IL-6 was prepared in accordance with manufacture specifications.

The binding stoichiometric ratio was determined by the intersection point of two straight lines extended from the initial linear part and the plateau part of the titration curve, respectively. The dissociation constant of the IL-6:QAB complex, K_{d}, was calculated using the formulation presented by Li and co-workers. (Li, J. J., Fang, X., and Tan, W., 2002. Biochem. Biophys. Res. Commun. 292, 31-40). Here the dissociation constant is expressed as K_{d}=C₀(1−θ)(r_{b}−θ)/θ where C₀ is the starting concentration of QAB, r_{b} is the molar ratio of IL-6 to QAB, and θ is the fraction of IL-6 bound to the immobilized QAB. θ can be determined using the equation θ=(I₀−I)/(I₀−I_{f}) where I₀ is the initial fluorescence intensity, I is the fluorescence intensity of the IL-6/QAB ratio in the titration curved, and I_{f} is the fluorescence intensity of the final data point the titration curve.

The conditions of the titration were as follows. Concentration [QAB]: 1 μM, Target Protein: Recombinant Mouse IL-6, carrier free, Target Protein Concentration, [IL-6]: 0-2.0 μM, Sample volume: 5 μL for QAB and IL-6. The temperature of the titration was 22° C. Seven QAB samples were hand spotted with pipettor onto an agarose-gelatin waveguide and dried in humidity chamber for about three hours prior to use. The excitation and emission fiber optics were configured such that the excitation occurred above the sample spot and the emission was detected orthogonally to the spot as it exited the waveguide. The detection point was 0.8 cm from the sample excitation spot. The samples were spotted sequentially on the waveguide. First was the QAB and then the addition of IL-6 onto the QAB spot. After generating a stable fluorescence signal the data point was recorded. The titration curve is created by the plot of fluorescence intensity versus the molar IL-6/QAB concentration ratio.

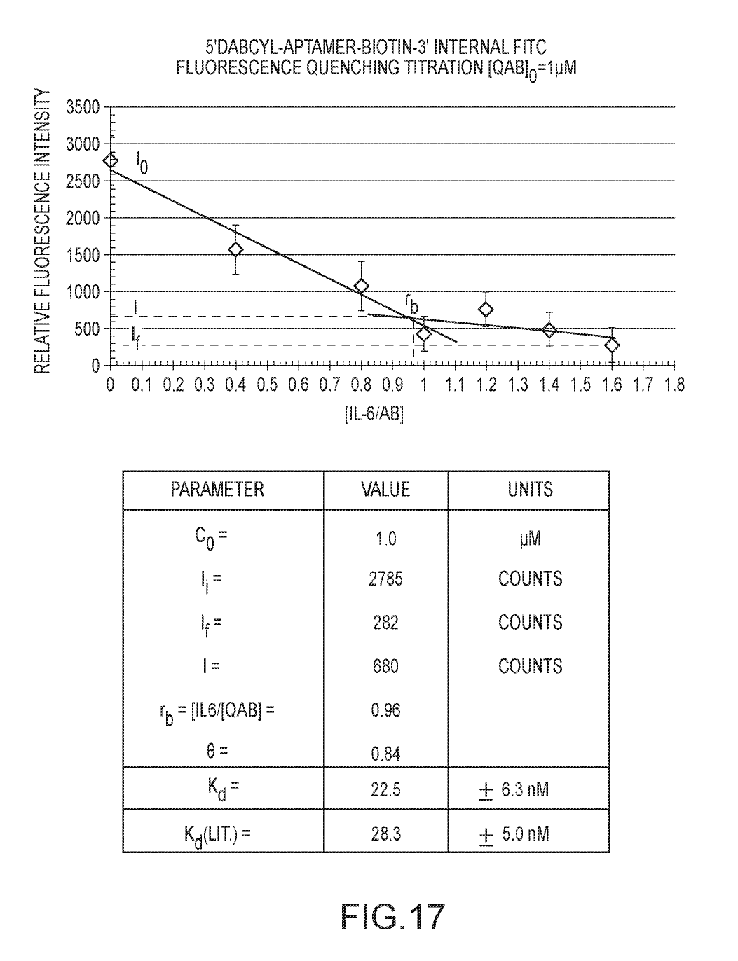

FIG. 17 shows the results of the titration. The fluorescence was linearly quenched by IL-6 over the first three or four data points of the titration, which indicated the IL-6 was mostly bound to the QAB. As more IL-6 was added the fluorescence curve flattens out with excess IL-6. Two linear lines are fitted to the data where the error bars are averages of four test runs. The intersection of the two lines gives the stoichiometric binding ratio r_{b} by extrapolating to the [IL-6]/[QAB] axis. In this manner, the binding ratio [IL-6]/[QAB] was found to be 0.96, which is consistent with an expected 1:1 binding ratio.

The dissociation constant for the IL-6-QAB complex, K_{d}, was determined using the equations above. In this example, the value of K_{d} for the IL-6/QAB complex was found to be 22.5±6.3 nM at 22° C. In comparison, the value reported by the manufacturer, BasePair Biotechnology, was 28.3±5.0 nM. The lower value reported here for the waveguide suggests that the IL-6 more tightly bound than the reported value in physiological buffer solutions. These data are summarized in a table in FIG. 17.

The following Embodiments are disclosed.

Embodiment 1. A product that is a medical device for collecting biofluids having a large area skin patch to collect, concentrate, and flow biofluids into a common, central flow channel.

Embodiment 2. The device of embodiment 1 that the concentrated biofluid flow from the common, central flow channel is directed to a biomarker detection chamber, and that fluid flow exits the biomarker detection chamber system, and out through the patch system.

Embodiment 3. The device of embodiment 1 that a sample chamber creates a high humidity environment in the microclimate next to the skin surface for sweat condensation.

Embodiment 4. The device of embodiment 1 that a sweat sample chamber generates and concentrates a liquid sweat sample containing a biomarker.

Embodiment 5. The device of embodiment 1 that a sweat sample chamber that contains a plurality of liquid flow channels that transports the liquid containing the biomarker to a biomarker recognition chamber.

Embodiment 6. The device of embodiment 1 that the liquid perspiration formed in skin pores can be induced and transported by a patch the covers the skin.

Embodiment 7. The device of embodiment 1 that the sample chamber material prevents water vapor diffusion out of the chamber and aids in the formation of liquid perspiration.

Embodiment 8. The device of embodiment 1 that the sample chamber contains channels made of hydrophobic and hydrophilic material.

Embodiment 9. The device of embodiment 1 that adjacent hydrophilic and hydrophobic bands create the liquid flow channels.

Embodiment 10. The device of embodiment 1 that liquid is transported in the hydrophilic material portion of the flow channel, and confined by the hydrophobic material.

Embodiment 11. The device of embodiment 1 that the hydrophilic material can be a wicking thread such as a wicking treated polyester or cotton fabric.

Embodiment 12. The device of embodiment 1 that the hydrophobic material can be a durable water repellent treatment, a fluorinated polymer, a siloxane polymer, a polyacrylic polymer, a polyurethane polymer that is coated or printed onto the hydrophilic material.

Embodiment 13. The device of embodiment 1 that the hydrophobic material is printed on the hydrophilic material in a geometric pattern or array to transport the liquid sample to a common inlet and outlet port.

Embodiment 14. The device of embodiment 1 that the sweat sample chamber and biomarker recognition chamber are integrated together into a single module to create a skin patch.

Embodiment 15. The device of embodiment 1 that the plurality of liquid flow channels can be used to transport saliva containing the biomarker to a biomarker recognition chamber.

Embodiment 16. The device of embodiment 15 that commercial saliva instruments can be used to collect saliva and deliver it to the liquid flow channels for analyzing biomarkers in saliva.

Embodiment 17. The device of embodiment 1 that a real time sweat flow rate and volume measurement system for sweat or saliva can be incorporated into the biosensor patch structure for aiding in determining the biomarker concentration.

Embodiment 18. The system of embodiment 17 that the real time sweat flow rate and volume measurement system is based on two fluorescent biopolymer waveguides positioned at different heights above the skin to measure the humidity gradient between the two waveguide points.

Embodiment 19. A wearable, disposable, biosensor device for continuous monitoring of specific biomarkers comprised of: A sweat sample chamber that creates a high humidity environment in the microclimate next to the skin surface. A sweat sample chamber that generates and concentrates a liquid sweat sample containing a biomarker. A sweat sample chamber that contains a plurality of liquid flow channels that transports the liquid containing the biomarker to a biomarker recognition chamber. A biomarker recognition chamber containing a biomarker binding site that is specific to the biomarker, and separates the biomarker from other sweat constituents. A biomarker binding site that generates an optical signal proportional biomarker concentration. A biopolymer based waveguide that guides the optical signal to a detection site. An analysis chamber that collects the optical signal from the biomarker binding site, and transduces the optical signal to an electronic signal when the biomarker is bound and separated from bulk sweat. An analysis chamber that wirelessly transmits diagnostic data on the detection and concentration level of a biomarker, risk patterns for the identified biomarker, and the occurrence of a health event to the patient, doctor and clinician.

Embodiment 20. The device of embodiment 19 that the liquid perspiration formed in skin pores can be induced and transported by a disposable patch the covers the skin.

Embodiment 21. The device of embodiment 19 that the sample chamber material prevents water vapor diffusion out of the chamber and aids in the formation of liquid perspiration.

Embodiment 22. The device of embodiment 19 that the sample chamber contains channels made of hydrophobic and hydrophilic material.

Embodiment 23. The device of embodiment 19 that adjacent hydrophilic and hydrophobic bands create the liquid flow channels.

Embodiment 24. The device of embodiment 19 that liquid is transported in the hydrophilic material portion of the flow channel, and confined by the hydrophobic material.

Embodiment 25. The device of embodiment 19 that the hydrophilic material can be a wicking thread such as a wicking treated polyester or cotton fabric.

Embodiment 26. The device of embodiment 19 that the hydrophobic material can be a durable water repellent treatment, a fluorinated polymer, a siloxane polymer, a polyacrylic polymer, a polyurethane polymer that is coated or printed onto the hydrophilic material.

Embodiment 27. The device of embodiment 19 that the hydrophobic material is printed on the hydrophilic material in a geometric pattern or array to transport the liquid sample to a common inlet and outlet port.

Embodiment 28. The device of embodiment 19 that the sweat sample chamber and biomarker recognition chamber are integrated together into a single module to create a disposable skin patch.

Embodiment 29. The device of embodiment 19 that the sweat sample contains a biomarker of pathophysiological importance such as inflammatory, neurohormones, myocyte injury, myocyte stress, and oxidative stress biomarkers.

Embodiment 30. The device of embodiment 19 that the biomarker recognition chamber contains a biomarker binding site that contains an aptamer immobilized on a biopolymer substrate to selectively bind the biomarker.

Embodiment 31. The device of embodiment 19 that the biomarker binding site substrate is configured as an optical waveguide.

Embodiment 32. The device of embodiment 19 that the biomarker binding site waveguide is constructed from common biopolymers such as agarose, gelatin, silk, and chitosan or other disposable biopolymers known to the art.

Embodiment 33. The device of embodiment 19 the biomarker binding site aptamer is modified to allow Forster Resonance Energy Transfer in either fluorescent signal-on or signal-off modes.

Embodiment 34. The device of embodiment 19 that the biomarker detection chamber contains an optical waveguide with a photon reflective element such as a notch groove, grating or reflective polymer film to reflect or scatter light perpendicularly into to the analysis chamber photodetector.

Embodiment 35. The device of embodiment 19 that the aptamer fluorescent signal generated at the biomarker binding site is coupled into the waveguide without extra optical components.

Embodiment 36. The device of embodiment 19 that the biomarker analyzer module contains a photodetector that detects the fluorescence co-linearly, perpendicularly, or at a preferred angle to the waveguide transmission direction, and transduces the optical signal to an electrical signal.

Embodiment 37. The device of embodiment 19 that the biomarker analyzer module provides a light source for the excitation of the fluorescent aptamer, and can be a LED, organic LED or laser diode.

Embodiment 38. The device of embodiment 19 that the biomarker analysis chamber photodetector is a photodiode or organic LED.

Embodiment 39. The device of embodiment 19 that the wireless data transmission module electronically connects to the biomarker analyzer module and contains circuitry and power supply to power the light source.

Embodiment 40. The device of embodiment 19 that the wireless transmission module contains firmware and software to control, monitor and perform data processing and analysis.

Embodiment 41. The device of embodiment 19 that the liquid collection chamber, biorecognition chamber, biomarker analyzer and wireless data transmitter can be worn as an adhesive bandage like patch.

Embodiment 42. The device of embodiment 19 that the plurality of liquid flow channels can be used to transport saliva containing the biomarker to a biomarker recognition chamber.

Embodiment 43. The device of embodiment 42 that commercial saliva instruments can be used to collect saliva and deliver it to the liquid flow channels for analyzing biomarkers in saliva.

Embodiment 44. The device of embodiment 19 that the wearable biofluid collection and biomarker recognition chambers can be worn on the skin to collect the biomarker in sweat and then removed to a free standing, detection chamber system for biomarker analysis having: An integrated porous adhesive skin patch that contains a sweat collector having a plurality of liquid flow channels, and a biomarker recognition chamber having an aptamer beacon and waveguide. A removable sweat collector chamber and biomarker recognition chamber system that can be freed from the adhesive patch and inserted into system support housing. A system support housing with smartphone and patch holder slots to position the smartphone and the collector and biomarker recognition chambers. Two optical light guide chambers that 1) guide the smartphone LED flash to excite the aptamer beacon on the waveguide, and 2) guide the waveguided fluorescent transmission to the smartphone camera for signal processing. A system support housing that isolates the stray and scattered light from reaching the detector. A smartphone application that performs data processing and analysis of the waveguided emission for generating and transmitting a status report of the individual's health state to the individual or doctor.

Embodiment 45. The system of embodiment 44 where the optical excitation is a separate and free standing LED or laser diode light source incorporated into the system housing,

Embodiment 46. The system of embodiment 44 where the LED or laser diode excitation light is guided to the biorecognition site using a light chamber.

Embodiment 47. The system of embodiment 44 where the fluorescent aptamer signal is guided to a smartphone camera for detection.

Embodiment 48. The system of embodiment 44 that the support housing contains a LED or laser driver circuit, power supply, and heat sink.

Embodiment 49. The device of embodiment 44 that the wearable, biosensor device can be used for non-continuous monitoring of specific biomarkers in sweat and saliva at specified time intervals throughout the day for at-home patient monitoring.

Embodiment 50. The system of embodiment 44 that the system can be used for detection of biomarkers in saliva.

Embodiment 51. The system of embodiment 50 that the system can be used for detection of biomarkers in saliva.

Embodiment 52. The device of embodiment 44 that a real time sweat flow rate and volume measurement system for sweat or saliva can be incorporated into the biosensor patch structure for aiding in determining the biomarker concentration.

Embodiment 53. The system of embodiment 52 that the real time sweat flow rate and volume measurement system is based on two fluorescent biopolymer waveguides positioned at different heights above the skin to measure the humidity gradient between the two waveguide points.

The terms and expressions employed herein are used as terms and expressions of description and not of limitation, and there is no intention, in the use of such terms and expressions, of excluding any equivalents of the features shown and described or portions thereof. In addition, having described certain embodiments, it will be apparent to those of ordinary skill in the art that other embodiments incorporating the concepts disclosed herein may be used without departing from the spirit and scope of the disclosure. Accordingly, the described embodiments are to be considered in all respects as only illustrative and not restrictive.

Each of the various elements disclosed herein may be achieved in a variety of manners. This disclosure should be understood to encompass each such variation, be it a variation of an embodiment of any apparatus embodiment, a method or process embodiment, or even merely a variation of any element of these. Particularly, it should be understood that the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this disclosure is entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, by way of example only, the disclosure of a detector should be understood to encompass disclosure of the act of detecting—whether explicitly discussed or not—and, conversely, were there only disclosure of the act of detecting, such a disclosure should be understood to encompass disclosure of a detecting mechanism. Such changes and alternative terms are to be understood to be explicitly included in the description. The previous description of the disclosed embodiments and examples is provided to enable any person skilled in the art to make or use the present disclosure as defined by the claims. Thus, the present disclosure is not intended to be limited to the examples disclosed herein. Various modifications to these embodiments will be readily apparent to those skilled in the art, and the generic principles defined herein may be applied to other embodiments without departing from the spirit or scope of the invention as claimed.

What is claimed is:

1. A wearable medical device for collecting biofluid, the device comprising:
a patch adapted to removably couple to a user's skin, the patch comprising at least one flow channel, at least one sample chamber, and at least one biomarker detection chamber; wherein
the sample chamber is adapted to create a high humidity environment adjacent the user's skin;
the at least one flow channel comprises a hydrophobic material and a hydrophilic material; and wherein
at least one of the sample chamber or the at least one flow channel is configured to conduct the biofluid towards the biomarker detection chamber.

2. The device of claim 1, wherein:
the hydrophilic material comprises a hydrophilic band; and
the hydrophobic material comprises a hydrophobic band proximal to the hydrophobic material whereby the at least one flow channel is configured to conduct biofluid towards the biomarker detection chamber.

3. The device of claim 2, wherein:
the at least one flow channel comprises a hydrophobic polyacrylic band deposited on a first side of a wicking treated hydrophilic polyester woven fabric.

4. The device of claim 3, further comprising:
a hydrophobic polyethylene film deposited on a second side of the wicking treated hydrophilic polyester woven fabric, the second side opposing the first side, whereby.

5. The device of claim 1, wherein:
the hydrophilic material comprises at least one of a wicking material, polyester, wool, polyvinylalcohol, hydrophilic polyurethane, polyolefin, polyamide, cellulose, polyacetate, polyacrylic, viscose, lyocell, rayon, cotton, or a material treated with a hydrophilic finish.

6. The device of claim 1, wherein:
the hydrophobic material comprises at least one of a durable water repellent treatment, a fluorinated polymer, a siloxane polymer, a polyolefin polymer, a sol gel material, a silane polymer, a polycarbonate, a polyethylene polymer, a polypropylene polymer, a polyester polymer, a polyacrylic polymer, or a polyurethane polymer

7. The device of claim 6, wherein the hydrophobic material is at least one of laminated, coated, or printed onto the hydrophilic material.

8. The device of claim 1 wherein:
the hydrophobic material is arranged on the hydrophilic material in a geometric pattern to conduct the biofluids.

9. The device of claim 1, wherein the sample chamber and the biomarker recognition chamber are unitary.

10. The device of claim 1, wherein:
the at least one flow channel comprises a plurality of liquid flow channels adapted to conduct saliva containing one or more biomarkers to the biomarker recognition chamber.

11. The device of claim 1, wherein:
the at least one flow channel forms a first cross-section flow area between the patch and the user's skin and a second cross-section flow area between the patch and the user's skin, the second cross-section flow area downstream of the first cross-section flow area; and wherein
and the first cross-section flow area is at least 10 times greater than the second cross-section area.

12. The device of claim 11, wherein:
the first cross-section flow area is at least 100 times greater than the second cross-section area

13. The device of claim 12 wherein:
the first cross-section flow area is at least 1,000 times greater than the second cross-section area.

14. The device of claim 13, wherein:
the first cross-section flow area is at least 10,000 times greater than the second cross-section area.

15. The device of claim 14, wherein:
the first cross-section flow area is at least 100,000 times greater than the second cross-section area.

16. The device of claim 1, further comprising:
a volume measurement system adapted to measure a flow rate of the biofluid.

17. The device of claim 1, further comprising:
a plurality of fluorescent biopolymer waveguides positioned at different distances from a first side of the patch, the plurality of fluorescent biopolymer waveguides adapted to measure a humidity gradient of the biofluid.

18. The device of claim 1, further comprising:
a first layer adapted to be positioned adjacent the user's skin, the first layer having the at least one flow channel positioned therein;
a second layer arranged on the first layer, the second layer adapted to prevent fluid from escaping the at least one flow channel;
a third layer arranged on the second layer, the third layer having the biomarker detection chamber; and
a fourth layer arranged on the third layer, the fourth layer having a hydrophilic material and adapted to promote capillary flow of the biofluid.

19. A method of collecting a biofluid, the method comprising:
coupling a wearable removable medical device for collecting biofluid to a user's skin for a period of time;
creating a high humidity environment adjacent the user's skin;
conducting a biofluid through at least one flow channel having a hydrophobic material and a hydrophilic material towards at least one biomarker detection chamber by way of capillary action.

20. A method of making a wearable medical device for collecting a biofluid, the method comprising:
providing a patch adapted to removably couple to a user's skin;
forming at least one flow channel in fluid communication with at least one sample chamber and at least one biomarker detection chamber in the patch; wherein
the at least one flow channel comprises a hydrophobic material and a hydrophilic material;
at least one of the at least one sample chamber or the at least one flow channel is shaped and positioned to conduct the biofluid towards the at least one biomarker detection chamber; and
the at least one sample chamber comprises the hydrophobic material, and is shaped and positioned to create a high humidity environment adjacent the user's skin.
